(19) United States
(12) Patent Application Publication
(10) Pub. No.: US 2026/0028618 A1
(43) Pub. Date: Jan. 29, 2026

(54) SAMPLE PROCESSING BARCODED BEAD COMPOSITION, METHOD, MANUFACTURING, AND SYSTEM

(71) Applicant: Bio-Rad Laboratories, Inc., Hercules, CA (US)

(72) Inventors: Adam McCoy, Hercules, CA (US); Kalyan Handique, Ann Arbor, MI (US); Jonathan Bell, Hercules, CA (US); Vishal Sharma, Hercules, CA (US); Sida Wang, Saline, MI (US)

(21) Appl. No.: 19/077,901

(22) Filed: Mar. 12, 2025

Related U.S. Application Data
(63) Continuation of application No. 17/109,704, filed on Dec. 2, 2020, now abandoned.
(60) Provisional application No. 62/945,006, filed on Dec. 6, 2019.

(51) Int. Cl. C12N 15/10; C12Q 1/6853; C12Q 1/686

(52) U.S. Cl. CPC C12N 15/1065; C12Q 1/6853; C12Q 1/686

(57) ABSTRACT

An embodiment of a composition for target material separation includes: a body and one or more molecules coupled to the body and structured for functionalization of the composition. In embodiments, each of the one or more molecules can include one or more of: a linker region; a polymerase chain reaction (PCR) segment or oligo binding region; one or more barcode region(s); a unique molecule identifier; a preparation-facilitating segment; an active segment; and a molecular scissor or cleavage region, wherein various regions can be coupled together in order to provide functionality to the composition. The invention(s) also cover manufacturing of the composition and various applications of use, in the context of target material capture (e.g., from single cells or other biological material).

### CROSS-REFERENCE TO RELATED APPLICATIONS

This application claims the benefit of U.S. Provisional Application No. 62/945,006 filed on 6 Dec. 2019, which is incorporated in its entirety herein by this reference.

### TECHNICAL FIELD

This invention relates generally to the cell capture and cell processing field, and more specifically to new and useful systems, methods, and compositions for sample processing barcoded beads for target material reactions.

### BACKGROUND

With an increased interest in cell-specific drug testing, diagnosis, and other assays, systems and methods that allow for individual cell isolation, identification, and retrieval are becoming highly desirable. Single cell capture systems and methods have been shown to be particularly advantageous for these applications. However, associated processes and protocols for single cell capture and subsequent analysis must often be performed in a particular manner and with a high precision in order to properly maintain the cells. Furthermore, efficient retrieval of target material from high density platforms is subject to many challenges. Additionally, compositions of materials can be improved significantly for applications involving capture and retrieval of target material in a manner that allows for single-cell analysis. As such, these processes can be time consuming for the user, can require extensive and iterative manual library preparation and selection processes, may not amenable to automation, and may thus result in damage to the cells (e.g., in terms of undesired loss of viability), high background noise rates, elevated false positive rates, or otherwise unreliable experimental results.

Thus, there is a need in the cell capture and cell processing field to create a new and useful system and method for sample processing and target material retrieval and minimize steps required in the library preparation of the target biomaterials, where some embodiments utilize molecular barcoding (e.g., through the use of barcoded oligonucleotides in the workflow typically delivered to a reaction environment involving functional particles). There is also a need for creating methods for streamlined manufacturing of described embodiments of barcoded beads in large quantities.

### BRIEF DESCRIPTION OF THE FIGURES

FIG. 1 depicts a schematic of an embodiment of a composition for target material reactions.

FIG. 2 depicts a schematic of an alternative embodiment of a composition for target material reactions.

FIG. 3 depicts schematics of embodiments of a linker molecule included in a composition for target material reactions.

FIGS. 4A-4C depict variations of a composition usable for mRNA capture to cDNA synthesis reactions or protein tagging interactions.

FIG. 5 depicts a variation of a composition including portions for simplification of library preparation operations.

FIG. 6A depicts a variation of a composition usable for mRNA capture to cDNA synthesis reactions.

FIG. 6B depicts a variation of a composition usable for protein tagging reactions.

FIGS. 6C-6E depict variations of the composition including thermolabile linker elements.

FIG. 7 depicts variations of a composition incorporating molecular scissor regions.

FIGS. 8A-8M depict variations of coupling functionalized molecules to a substrate.

FIGS. 9A and 9B depict variations of a composition usable for ATAC-seq operations.

FIGS. 9C-9E depict variations of a composition with restriction sites.

FIG. 10 depicts a flowchart of an embodiment of a method for ATAC-seq.

FIG. 11 depicts a flowchart of a method for manufacturing a composition.

FIG. 12A depicts a flowchart of a variation of a method for manufacturing particles of a composition.

FIGS. 12B and 12C depict variations of a step for manufacturing a composition.

FIG. 13 depicts variations of synthesis of an oligonucleotide molecule.

FIG. 14 depicts a variation of synthesis of a portion of an oligonucleotide molecule.

FIG. 15 depicts detailed steps of a variation of synthesis of a portion of an oligonucleotide molecule.

FIGS. 16A-16E depict variations of synthesis of a set of oligonucleotide molecule with unique barcodes coupled to a particle.

FIGS. 17A-17B depict alternative variations of synthesis of a set of oligonucleotide molecule with unique barcodes coupled to a particle.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Benefits

The invention(s) described can confer several benefits over conventional systems, methods, and compositions.

The invention(s) confer(s) the benefit of providing non-naturally occurring compositions for facilitating capture, extraction, and/or retrieval of target biological material from a sample, while providing barcoding for each biomarker molecule retrieved from a partition of a sample which may be discrete single cells in the sample. Such compositions can include materials that have been modified from their natural states (e.g., in terms of providing structural differences from natural compositions). Furthermore, the invention(s) relate to combinations of materials, where the combinations of materials are non-naturally occurring (e.g., there is no naturally occurring counterpart to the compositions described and claimed).

The invention(s) also include novel compositions of base material and chemistry of components, to produce simplifications in library preparation processes.

The invention(s) also include novel compositions with cleavable sites that allow for separation of target material, with the ability to monitor cleavage and/or quantify components processed from a biological sample.

The invention(s) also confer(s) the benefit of providing mechanisms for efficient retrieval of target material (e.g., beads, cells, released nucleic acid material, etc.) from high-aspect wells of a high-density capture platform. Retrieval is typically difficult and non-efficient in this scenario due to close packing of wells of the capture platform. Retrieval mechanisms described also subject target material to acceptable amounts of shear and other potential stresses that would otherwise obstruct downstream processing steps.

The invention(s) also confer(s) the benefit of providing methods for manufacturing beads for capturing target molecules and/or molecules coupled to a substrate (e.g., chamber wall), where the molecules include a set of unique barcodes that can be detected for sample processing.

The invention(s) also confer(s) the benefit of reducing burden on system operators in relation to target material retrieval processes from wells, where standard processes can be inefficient/labor intensive.

The invention(s) also confer(s) the benefit of increasing the efficiency at which target material is retrieved (and non-target material is not retrieved). Selective retrieval efficiency can thus reduce downstream costs in relation to processing reagent and other material costs (due to reduced volumes needed), processing burden, and improved signal to noise ratios. For instance, the invention(s) can enable a system operator to purchase smaller volumes of reagents, reduce the number of splits required for successful amplification of target molecules and obviate the need for doing SPRI-based clean-up and size selection of target oligonucleotide products from other oligonucleotide tags that do not contain products but get carried over from one process step to the next. Such improved recovery of target products and reduction of carryover of non-target products can also reduce the complexity of data analysis and also provide more useable data pertaining to the desired biomarker analysis as well. This can function to save costs, reduce reagent waste, or have any other suitable outcome.

The invention(s) also confer(s) the benefit of providing greater sequencing depth with respect to desired target, due to greater numbers of target reads provided by the compositions, methods, and systems described.

The invention(s) also confer the benefit of enabling at least partial automation of the protocols involved in single cell capture, target material retrieval, and subsequent processing. For instance, a human operator user can be removed from part or all of the method. Furthermore, the system(s) and/or method(s) can enable better accuracy in performance of a protocol over conventional systems and methods. Some of these inventions are also much more amenable to full automation with a liquid handling robot.

Additionally or alternatively, the invention(s) can confer any other suitable benefit.

### 2. Functional Bead Composition

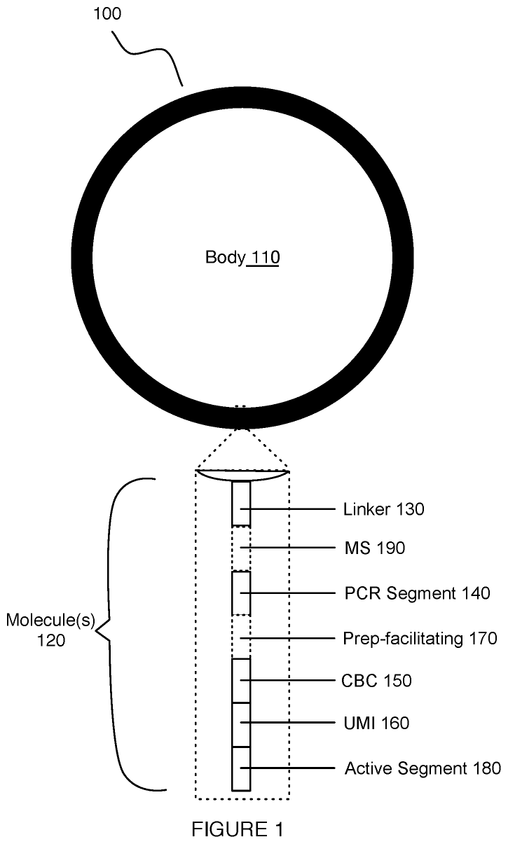

As shown in FIG. 1, an embodiment of a composition 100 for target material separation includes: a body 110 and one or more molecules 120 coupled to the body 110 and structured for functionalization of the composition 100. In embodiments, each of the one or more molecules 120 can include one or more of: a linker region 130; a polymerase chain reaction (PCR) segment or oligonucleotide binding region 140; one or more barcode region(s) 150; a unique molecule identifier 160; a preparation-facilitating segment 170; an active segment 180; and a molecular scissor or cleavage region 190, wherein various regions can be coupled together (e.g., in sequence) in order to provide functionality to the composition. In applications, the composition 100 can be provided as a set of functionalized particles each with a set of coupled oligonucleotide molecules for various assays configured to facilitate extraction operations, amplification processes, size-based purification processes, binding processes, release and retrieval processes, and other reactions (e.g., molecular reactions) for single-cell analyses.

The composition 100 can be configured to operate with systems configured to perform single-cell analyses, in manual, semi-automatic, and/or automatic operation modes. Embodiments, variations, and examples of such systems are described in one or more of: U.S. application Ser. No. 13/557,510 titled “Cell Capture System and Method of Use” and filed on 25 Jul. 2012, U.S. application Ser. No. 14/289,155 titled “System and Method for Isolating and Analyzing Cells” and filed on 28 May 2014, U.S. application Ser. No. 15/422,222 titled “System and Method for Isolating and Analyzing Cells” and filed on 24 Feb. 2017, U.S. application Ser. No. 15/815,532 titled “System and Method for Retrieving and Analyzing Particles” and filed on 16 Nov. 2017, and .S. application Ser. No. 16/115,059 titled “System and Method for Isolating and Analyzing Cells” and filed on 28 Aug. 2018 which are each incorporated in their entireties by this reference.

The composition 100 can be configured for processes and reactions associated with one or more of: a reverse transcription reaction (RT-reaction), immunochemistry, DNA reactions, mRNA FISH reactions, proximity ligation reactions, bridge amplification reactions, catalytic enzymatic reactions, hybridization reactions, restriction digestion reactions, amplification reactions (e.g., mRNA and/or DNA PCR), and other suitable reactions. Such reactions can be performed on-chip and/or off-chip, where embodiments, variations, and examples of microfluidic chips for single-cell analyses are described in U.S. application Ser. No. 13/557,510 titled “Cell Capture System and Method of Use” and filed on 25 Jul. 2012, U.S. application Ser. No. 14/289,155 titled “System and Method for Isolating and Analyzing Cells” and filed on 28 May 2014, U.S. application Ser. No. 15/422,222 titled “System and Method for Isolating and Analyzing Cells” and filed on 24 Feb. 2017, and U.S. application Ser. No. 15/815,532 titled “System and Method for Retrieving and Analyzing Particles” and filed on 16 Nov. 2017, which are each incorporated in their entireties by this reference.

### 2.1 Functional Bead Core

The body 110 functions to provide a substrate to which the one or more molecules 120 can be coupled to, in order to provide functionalization for the composition with respect to implementation of respective assays and reactions.

In relation to morphology, the body 110 can have the form of a microsphere. Alternatively, the body 110 can have the form of a non-spherical (e.g., ellipsoidal, prismatic, polyhedral, amorphous, etc.) body, where a cross section taken through the body 110 is non-circular. However, the body 110 can alternatively have another suitable form. In relation to dimensions, the body 110 can have a diameter (or characteristic width) from 5-50 microns, with a tolerance of +0.05 to 5 microns. Additionally, the uniformity of the body 110 across a population of particles can enable a desired retrieval efficiency behavior upon completion of various steps of an intended single cell process. In a specific example, the body 110 has a diameter of 20 microns±1 micron; however, variations of the example body 110 can have other morphology.

In embodiments, the body 110 has a characteristic dimension configured such that only a single body 110 of the composition 100 can enter a well of the chip described above, along with a single target cell, in order to co-localize and co-capture the single cell-particle pair within an individual well. However, the body 110 of the composition 100 can have another suitable characteristic dimension configured for other microfluidic or non-microfluidic assay applications.

In relation to density, the body 110 is configured to have a density greater than the density of process liquids intended for use with the composition 100 (e.g., in relation to specific reactions or assays), such that the composition 100 settles within the process liquid(s) by gravity during operation. In an embodiment, the density of the body 110 is greater than 1.02 g/cm³, however, the body 110 can have other suitable densities in variations. For instance, the body 110 can be configured to be of the same density as an intended process liquid in some embodiments (e.g., in order to facilitate steps where the body 110 is desired to be carried with flow of the process liquid). In still other embodiments, the body 110 can be configured to be buoyant relative to a process liquid, such that the body 110 is buoyant and can be used for separation of target or non-target material of a sample.

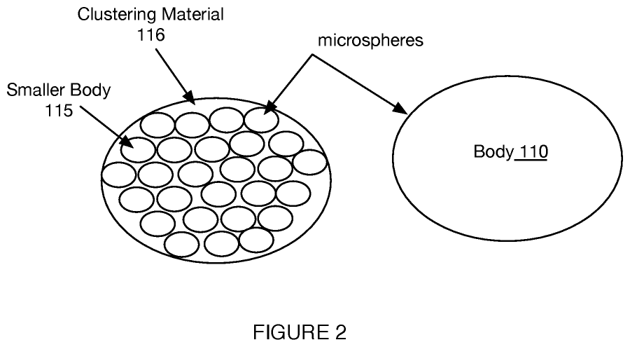

In relation to density and morphology, the body 110 can be a continuous body (e.g., at micron scale, at nanometer scale, at sub-nanometer scale). Alternatively, a variation of which is shown in FIG. 2, the body 110 can be composed of a cluster of smaller bodies 115 (e.g., having morphology scaled down from the macroscopic morphology of the body 110, having other morphology). Such a configuration can provide greater overall surface area due to the aggregation of surfaces of the smaller bodies 115, can produce macroscopic behavior (e.g., in terms of approximate rigidity/other mechanical properties) of a single body for oligonucleotide synthesis, and/or can be dissolved after use in an assay (e.g., after capture) to provide desired surface chemistry behavior. The cluster of smaller bodies 115 can be surrounded by (e.g., encased in) a clustering material 116 that can temporarily or “permanently” maintain the cluster morphology of the cluster. In examples, the clustering material 116 can include a hydrogel, where the hydrogel has suitable properties (e.g., in terms of crosslinking, in terms of dissolvability, in terms of porosity, in terms of density, in terms of thermal properties, in terms of optical properties, in terms of charge, in terms of composition, in terms of mechanical properties, in terms of other physical properties, etc.) for intended use of the composition. In a related application of use, the clustering material 116 can maintain clustered morphology of the smaller bodies 115 during a phase of use in an assay, and can then be dissolved or otherwise removed in order to transition the smaller bodies 115 to an non-clustered state (e.g., to provide improved access to surface chemistry of each of the smaller bodies.

In an example, a composite microsphere made of a number of small microspheres (e.g., having 0.5 micrometer diameters) reacted to the surface of a larger microsphere (e.g., having a 19 micron diameter), such that the composite microsphere had a total diameter of 20 micron but the surface area of the surface of the composite particle was significantly enhanced by the presence of the smaller microspheres or presence of certain reactive groups ordered in a specific pre-designed array.

In embodiments, the base materials and surface properties can be different to offer significant flexibilities of performance. For example, the bigger microsphere may be a hard material while the small microspheres could be of hydrogel. In another example, the larger microspheres can be non-magnetic but the smaller microspheres can be magnetic. In another example, the larger microsphere is magnetic and the smaller microspheres are magnetic or paramagnetic. In another example, the larger microsphere can be made of transparent material while the smaller microspheres may be of optically (e.g., brightfield or fluorescent) coded. In another example, the larger microsphere can be made dissolvable while the smaller microsphere are non-dissolvable. Another embodiment of a composite microsphere could include a set of base hard microspheres coated with a thin (e.g., 1-3 micron layer) of hydrogel or other material(s) providing increased surface area of reactions. Such an innovative microsphere would also provide an added advantage of allowing biomarkers of certain size to permeate into the microsphere to part-take in a specific reaction. Yet another example of composite microspheres could include solid particles (e.g., 20 micron diameter) with micro-tunnels (e.g., 0.1-2 micron diameter) that span from the surface of the composite microsphere to the center of the microspheres. In some cases, these micro-tunnels could go across the diameter of the entire particle. In still other embodiments the micro-tunnels are pores which increase the total surface area of the composite materials. In yet another embodiment, the large microsphere may have a thin coating on the surface that has a different functional composition compared to the composition inside. The top surface may be cross-linked but the inside material may be soft or dissolvable.

In variations, each of the smaller bodies 115 can be the same in properties and composition; however, in other variations, one or more of the smaller bodies 115 can be configured to have different properties, compositions, and distributions within the cluster (e.g., from the core to the surface), in order to provide different functionality for different portions of an assay or reaction. For instance, a first region (e.g., surface) of the cluster can have a first set of properties, composition, and/or surface chemistry to perform a first part of an assay or reaction, be dissolved or otherwise removed, and then a second region (e.g., core) of the cluster can have a second set of properties, composition, and/or surface chemistry to perform a second part of an assay or reaction.

In a specific example, a set of approximately 750 smaller bodies 115 each composed of polystyrene with divinylbenzene crosslinking (PS-DVB) having a diameter of 1 micron (with suitable tolerance) are clustered in a dissolvable hydrogel to provide a gross diameter of 20 microns, with overall surface area ˜7.5 times that of a single contiguous 20 micron particles. In another example, the body 110 can be composed of a hydrogel where the smaller bodies are made up of poly-acrylamide matrix and the clustering material comprises a disulfide crosslinking agent (e.g., BAC). However, variations of the example can be configured in another suitable manner.

In relation to thermal properties, the body 110 is configured to operate between a lower temperature limit (e.g., associated with low temperature reactions and processes, associated with storage, etc.) and an upper temperature limit (e.g., associated with high temperature reactions and processes). In specific examples, the lower temperature limit is from −20° C. through 4° C. (e.g., for cold storage), and the upper temperature limit is from 90° C. through 120° C. (e.g., for denaturation reactions). However, the body 110 can be configured for other operating temperatures.

In relation to physical properties, the body 110 is configured to maintain structure in solution (e.g., in buffer during storage, in solution during performance of an assay). As such, the body 110 is configured to be non-swelling and non-leaching. However, in alternative embodiments, the body 110 can be configured to swell a desired amount (e.g., in relation to achieving a desired size or morphology for processing or use in an application), configured to leach certain compounds (e.g., process reagents) for performance of an assay, and/or to dissolve in a desired manner during performance of an assay or other process. In yet another embodiment, the particle may have well-defined tailored swellability such that its use in specific buffer and/or physical conditions allows the particles to easily enter a microwell but may be trapped in the microwell under specific buffer conditions. Further in relation to physical properties, the body 110 can be configured with a desired degree of hydrophilicity (e.g., on a spectrum from hydrophilic to hydrophobic) in relation to performance of an assay or other process. In relation to surface properties associated with fluid contact, the body 110 can be configured to have a desired wettability (e.g., in terms of contact angle, etc.). Variations of the body 110 can thus have a suitable type of crosslinking (e.g., chemical crosslinking, physical crosslinking, etc.) and percentage of crosslinking (e.g., from 1-10% crosslinking for acrylamide, 30-99% crosslinking for other materials, another suitable range of crosslinking), to provide a desired level of stability in conditions of use.

In relation to other surface properties, the body 110 can be configured with a desired porosity (e.g., 200-2000A, etc.). The body 110 can additionally or alternatively be configured with a desired loading density (LD), in order to enable achievement of a suitable linker density (e.g., by providing points of attachment on the body 110 to provide more robust detectible signals during use), where additions to the body 110 are described in more detail in Section 2.2 below. Furthermore, the body 110 can include surface groups (e.g., hydroxyl groups, amine groups, carboxyl groups, sulfide groups, silanol groups, etc.) for coupling of linker molecules described in Section 2.2 below. In examples, desired loading density (LD) can be as low as 1 umol/g or as high as few hundred umol/g of functional group density.

In relation to magnetic properties, the body 110 can be configured to respond to magnetic fields (e.g., in relation to assays involving separation and/or retrieval of target or non-target material). Certain regions (e.g., a core region) of the body 110 can be magnetic (e.g., magnetic, paramagnetic, etc.), and certain regions (e.g., a shell region) of the body 110 can be non-magnetic in variations of the body 110. In relation to surface properties, the body 110 can be configured with or without charge, in order to facilitate binding to target material, or to facilitate fabrication involving molecules with functionality.

In relation to optical properties, the body 110 can be configured to be non-fluorescent (e.g., so as to not interfere with optical-based detection assays). However, in variations, the body 110 can be configured to be optically detectable (e.g., via a non-fluorescent modality, via a fluorescent modality, via an infrared detection modality, via a thermal detection modality, etc.), for instance, for tracking purposes.

In relation to mechanical properties, the body 110 can be configured to have a desired hardness (e.g., measured on the Mohs scale, measured on another hardness scale), in order to retain a desired level of hardness during applications of use. Additionally or alternatively, the body 110 can be configured with desired mechanical properties associated with one or more of: rigidity, elastic behavior (e.g., in terms of moduli, in terms of plastic and elastic deformation, etc.), viscoelastic behavior, fatigue resistance, fracture resistance, shear strength, compressive strength, tensile strength, rheological behavior (e.g., under conditions of wear), and other mechanical properties.

In relation to composition, the body 110 can be composed of one or more of: polystyrene, polystyrene-divinylbenzene, polymethylmethacrylate (PMMA), silica, silica-gel, non-porous glass, porous glass, coated glass, agarose, acrylamide, polyacrylamide, iron, steel, or ceramic materials and/or a combination of one or more suitable materials. As noted above and below, different regions of the body 110 can be composed of different materials (e.g., a core region can be composed of a first material and a shell region can be composed of a second material). In some embodiments there may be multiple regions either as multiple shell regions, or in other configurations such as amorphous or ordered spatial arrangements.

Specific examples of the body 110 are composed of polyacrylamide (e.g., as described in more detail below, silica (e.g., silica gel), polystyrene, or PMMA, 15-25 microns in diameter (e.g., where smaller diameters allow for minor swelling in a manner that is still appropriate for use within microfluidic structures), with a surface porosity from 80-1500A, with between 20% and 80% crosslinking (e.g., Polystyrene with 60% crosslinking by divinylbenzene or Polystyrene with 80% crosslinking by divinylbenzene) for polymeric beads, with surface groups (e.g. amine groups, hydroxyl groups, silanol groups) for coupling of linker chemistry (e.g., C18 tag linker), and polyethylene glycol (PEG) functionalization for reaction efficiency. Variations of the specific examples can have magnetic (e.g., magnetic, paramagnetic) cores or shells to allow for magnetic functionality (e.g., for separation and retrieval).

### 2.2 Functional Molecule(s)

As shown in FIG. 1, the composition 100 also includes one or more molecules 120 coupled to the body 110 and structured for functionalization of the composition 100. In embodiments, each of the one or more molecules 120 can include one or more of: a linker region 130; a polymerase chain reaction (PCR) segment 140; a barcode region 150; a unique molecule identifier 160; a preparation-facilitating segment 170; an active segment 180; and a molecular scissor region 190, wherein various regions can be coupled together (e.g., in sequence) in order to provide functionality to the composition. The one or more molecules 120 can function to provide desired chemistries (e.g., binding chemistries) for different reactions or processes, and in variations, inclusion of specific oligonucleotides in the one or more molecules can adapt the one or more molecules for mRNA binding, binding of CITE-sequencing probes, oligonucleotide labeled antibodies, oligonucleotide labeled peptides, oligonucleotide labeled lipids, oligonucleotide labeled metabolites, modified genomic DNA, unmodified genomic DNA, DNA ATAC sequencing, g, Hi-C sequencing, cut-n-tag sequencing, bridge amplifications, proximity ligations, other molecular reactions, other protein-tagging operations, and/or other reactions. Furthermore, the one or more molecules can be adapted for facilitation of library preparation operations, by inclusion of regions (e.g., specific adaptors, primers) for various sequencing platforms (e.g., next generation sequencing platforms, Illumina™ sequencing platforms, etc.). As such, the one or more molecules 120 can simplify manual or automatic steps associated with sequencing or other reactions, by incorporation of specific oligonucleotide segments.

In embodiments, the one or more molecules 120 can include a single molecule, a set of identical molecules, or a set of different molecules (e.g., a first and a second molecule, a plurality of different molecules) distributed across a body 110. For instance, in reactions involving mRNA capture and cDNA synthesis, the one or more molecules 120 can include oligonucleotide molecules having a first sequence for mRNA binding, and having a second sequence associated with generation of complementary cDNA strands. Similarly, in reactions involving binding of protein tags, the one or more molecules can include molecules having a first sequence for detecting antibody binding through detecting tagging of antibodies with an oligonucleotide tag, and molecules having a second sequence for synthesis. In another embodiment, different sets of molecules for providing forward as well as reverse primers may be present in the one or more molecules 120 to allow for bridge amplifications to amplify certain nucleic acid fragments from single cells that are initially bound to the microspheres. Relative proportions of various forward or reverse primers may be adjusted such that only cDNA of certain sizes are maximized during bridge amplification (e.g., for example products less than 600 base pairs or more than 300 base pairs). However, the sequences of the one or more molecules 120 can be adapted for other reactions and processes, variations of which are described below in relation to different structural features of the one or more molecules 120. Binding groups may also be present in 120 in certain proportion for enzymes to be tethered to the microsphere during enzymatic reactions such that these enzymes can process and create reaction products for mRNA to reach only a certain size or prevent products to be more than certain base sizes. Alternatively, the structural features may exclude certain enzymes (e.g., nucleases or restriction enzymes) or other functional moieties from close proximity to the body 110 in order to adjust the size of the retained molecules to a desirable size (eg., anything longer than 300 bp is digested to smaller size).

### 2.2.1 Molecule-Linker

As shown in FIG. 1, the body 110 can include a set of linkers including linker 130, wherein the linker 130 functions to control density and spacing of the one or more molecules 120 coupled to the body 110, in a manner that provides a sufficient number of molecules/sites for reactions to occur. The set of linkers also functions to control density and spacing of the one or more molecules 120 in a manner that prevents molecules at the surfaces of the body(ies) from folding or otherwise forming undesired structures (e.g., secondary structures, tertiary structures, etc.) or in other embodiments controls density in such a way it promotes such structures.

In embodiments, the number of linkers in the set of linkers is configured to be greater than the number of target molecules per single cell being targeted for binding reactions, In one example, the number of target molecules per cell is on the order of 0.5 to 1 million molecules or molecule fragments; thus, in the example, the set of linkers can include 107-1010 linkers for positioning 107-1010 full-length oligonucleotides per body 110, wherein an excess of full-length oligonucleotides result in more mRNAs (or other molecules) captured during a reaction. However, the set of linkers can include other numbers of linkers in other embodiments.

In embodiments, the linker 130 comprises a branched linker configured to provide suitable density of oligonucleotide molecules at the surface of the body 110, and to provide suitable spacing between adjacent oligonucleotide molecules. In variations, the branched linker is a dendrimer (e.g., symmetric dendrimer, asymmetric dendrimer, doubler, trebler, labelled, non-labelled, etc.), that provides branching with nodes of attachment. In one variation, the dendrimer can be a y-shaped dendrimer that includes a source node (e.g., for attachment at a region of the body 110 or proximal to the body 110), and two terminal nodes (e.g., for attachment to functional oligonucleotide molecules of the one or more molecules 120 or for attachment to subsequent dendrimers distal to the body 110). In a specific example, the branched linker is a symmetric doubling phosphoramidite dendrimer; however, variations of the specific example can use another core chemistry (e.g., carbosilane, thiolated, etc.) and structure. As such, in other variations, the dendrimer can have any other suitable number of attachment points, chemistry, and/or structure, to provide spacing and sites of coupling for oligonucleotide molecules to the body 110.

Furthermore, the branched linker can be configured for selectable attachment (e.g., with functional groups specific to specific chemistries) and/or selectable cleavage (e.g., for release of oligonucleotide segments, such as molecular scissors, during processing).

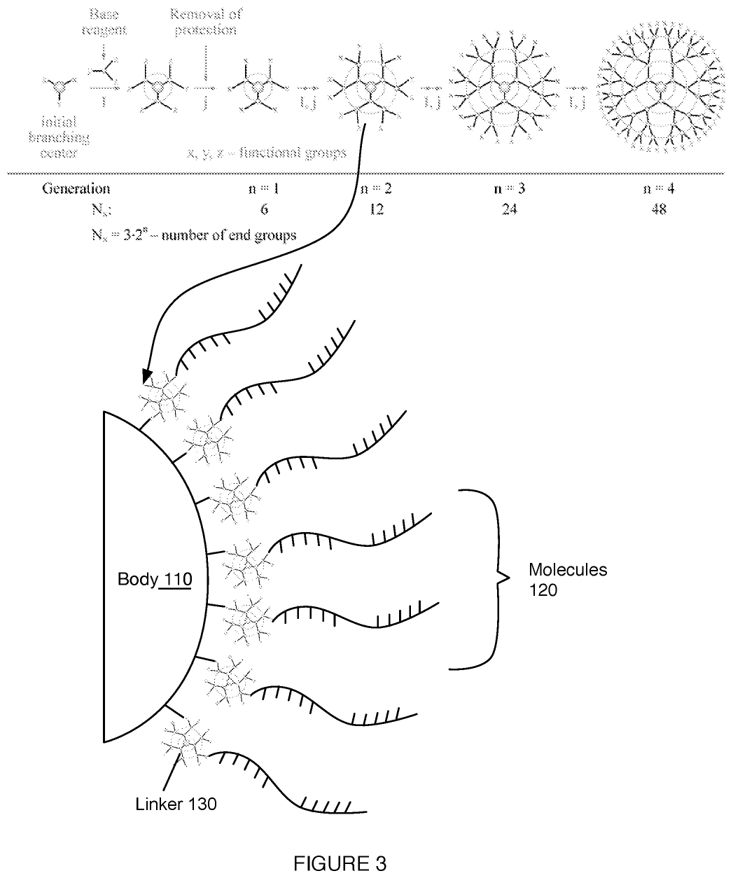

As shown in FIG. 3, a dendrimer useful as a linker can be formed by starting with an initial branching center, coupling a set of base reagents to the initial branching center, and sequentially adding generations of base reagents until a desired dendrimer size and number of terminal branches (e.g., an exponential of the number of generations) is achieved. The type of base reagent functional group, number of generations, and molecular weight can produce a hydrodynamic diameter corresponding to a desired diameter corresponding to oligonucleotide helix width (e.g., ˜2 nm), in order to achieve a desired density of oligonucleotide molecules coupled to the body 110, by way of the design of the linkers. However, the final diameter (or other characteristic dimension) of the dendrimeric linkers can be configured to match another design constraint or configured in another suitable manner.

### 2.2.2 Molecule-PCR Segment(s)

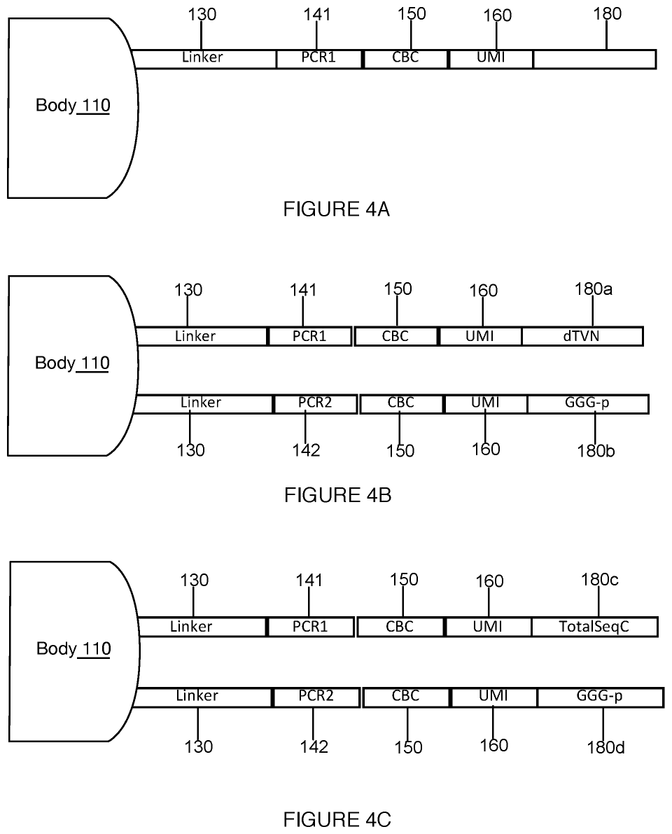

As shown in FIG. 1, each of the one or more molecules can further include one or more polymerase chain reaction (PCR) segments 140 configured for performance of a PCR-associated reaction (e.g., amplification). The PCR segment(s) can include PCR primers for performance of a PCR reaction. As indicated above in relation to different types of nucleic acid-associated and protein-associated reactions (and shown in FIGS. 4A through 4C, the PCR primer(s) used for different sequences of the one or more molecules 120 can be identical or different from each other. For instance, in a first variation, a first portion of the one or more molecules 120 can include a first PCR primer segment 141 associated with a first phase of a reaction (e.g., mRNA binding, binding of antibodies, binding of other protein tags, etc.), and a second portion of the one or more molecules 120 can include a second PCR primer segment 142 associated with a second phase of a reaction (e.g., cDNA synthesis, other synthesis, other tagging, other binding, etc.).

In other variations, the PCR segment(s) 140 can additionally or alternatively include a PCR handle segment 143 that is detectable and configured for quality control of the composition. However, variations of the one or more molecules 120 can additionally or alternatively omit the PCR handle segment 143.

In embodiments, the PCR segment(s) 140 are coupled directly to a terminal portion (or other portion) of one of the set of linkers 130. However, in other variations, the PCR segment(s) can be coupled relative to other portions of an oligonucleotide molecule in another manner.

In embodiments, the PCR segment(s) 140 can have from 5-30 bases and can include custom or non-custom primers; however, in alternative variations the PCR segment(s) 140 can have other suitable numbers of bases.

### 2.2.3 Molecule-Barcode Region and Unique Molecule Identifier (UMI)

As shown in FIG. 1, each of the one or more molecules 120 can include a barcode region 150, which functions to enable unique identification of biological material (e.g., cellular material) processed or derived from (e.g., synthesized from) using the one or more molecules 120 of the composition 100. The barcode region 150 can be configured to reduce noise in relation to detected signals and usable reads (e.g., in relation to assignment of sequencing reads to the correct barcode and reduction of wasted reads). In relation to the method 400 of manufacture described in more detail below, accuracy of the barcode region 150 across all molecules of coupled to a particular body 110 (in relation to minimizing unintentional deletions, substitutions or additions) can thus result in low error rates with respect to false positives (e.g., matching of signals to an incorrectly barcoded molecule).

As shown in FIG. 1, the barcode region 150 can be coupled to the PCR segment 140 (e.g., distal to the PCR segment 140 relative to the body 110) or can alternatively be coupled to another portion of a molecule of the one or more molecules 120.

The barcode region 150 can include one or more barcode segments, where manufacture and assembly of the barcode segments are described in more detail in Section 4 below. In some variations the barcode segment may include portions used for assembly (e.g., a handle such as a ligation handle or PCR extension handle) which can alternately be used as portions of barcode or independently from the barcode segments.

In variations, each barcode segment can be from 2-20 nucleotides long; however, in alternative variations, each barcode segment can have another suitable length. Preferably, each barcode segment has a Hamming distance (e.g., number of substitutions required to make two strings of nucleic acids identical) greater than 2; however, in alternative variations, the barcode segments can have another suitable Hamming distance. Furthermore, each barcode segment can be configured to not end in GG (or other sequences that are less suitable for specific sequencing platforms); however, the barcode segments can be configured in another suitable manner. The barcode region 150 can be constructed from one or more segments to create 1-100 million unique barcodes of suitable length; however, variations can produce other suitable numbers of unique barcodes. In a specific example, the barcode segments are selected from a set of 875 (or more) 7-mers having a Hamming distance of 2 without termination in GG bases, where the sequences are non-naturally occurring. In the specific example, the barcode region is composed of multiple segments that, when assembled together, create 50 million unique barcodes. However, variations of the specific example can be configured in another suitable manner.

As shown in FIG. 1, each of the one or more molecules 120 can include a unique molecule identifier (UMI) 160 which functions as a molecular tag to allow sequencing platforms (e.g., next generation sequencing platforms) to identify the input molecule being processed. Each molecule of the one or more molecules 120 can have a single UMI or multiple UMIs. Furthermore, the UMI 160 can be coupled to the barcode region 150 (e.g., distal to the barcode region 150) as shown in FIG. 1, or in another position along a molecule of the one or more molecules 120.

### 2.2.4 Molecule-Preparation-Facilitating Segments

As shown in FIG. 1, each of the one or more molecules 120 can optionally include one or more preparation-facilitating segment(s) 170, which functions to simplify or otherwise reduce processing steps associated with certain operations.

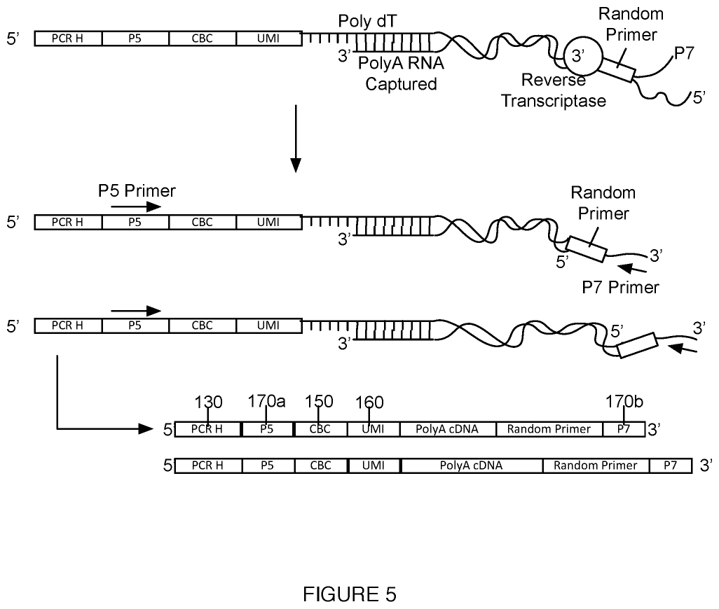

In one variation, as shown in FIG. 5, the preparation-facilitating segment(s) 170 can be configured to simplify library preparation steps by incorporation of sequences of molecules that have to typically be implemented in separate steps (e.g., in otherwise a manual-manner). In more detail, a molecule of the one or more molecules 120 can include a first preparation-facilitating segment 170a associated with a P5 adapter (e.g., for Illumina™ flow cells), wherein, in some variations, the first preparation-facilitating segment 170a includes sequences for a partial P5 adapter and associated index. In variations, the first preparation-facilitating segment 170a can be coupled to the barcode region 150 (e.g., proximal to the body 110, another suitable region). The molecule of the one or more molecules 120 can also include a second preparation-facilitating segment 170b associated with a P7 adapter (e.g., for Illumina™ platforms and configured for cDNA synthesis), which may be added during the same step or in a reverse transcription process or other separate step, wherein, in some variations, the second preparation-facilitating segment 170b includes sequences for a random primer configured to randomly bind to a target mRNA molecule closer to the 3′ end of the mRNA molecule and prevent extension on 5′ end of the mRNA molecule. As such, during reverse transcription the cDNA strand will terminate adjacent to the random primer segment. A ligase enzyme will then ligate the random primer with attached facilitating segment 170b to the cDNA strand. Subsequent amplification with P7 and P5 primers would result in a sequenceable fragment without the need for fragmentation during indexing. Such a configuration would also produce exponential amplification of signal but only linear amplification of noise, thereby significantly improving the signal-to-noise ratio (SNR). As such, incorporation of the preparation-facilitating segments 170a, 170b can collapse multiple steps into a single step, and streamline a cleanup process that would otherwise have to be performed (e.g., given that the desired product would be coupled to the composition 100 after use of the composition).

However, in other variations, the preparation-facilitating segment(s) 170 can additionally or alternatively include other sequences configured to reduce steps (e.g., manual steps) associated with operations (e.g., for specific platforms, for specific processes, etc.).

### 2.2.5 Molecule-Active Segment

As shown in FIG. 1, each of the one or more molecules 120 can optionally include an active segment 180, which functions to enable performance of a desired process (e.g., binding interaction to enable tagging or synthesis associated with nucleic acid molecules, proteins, etc.).

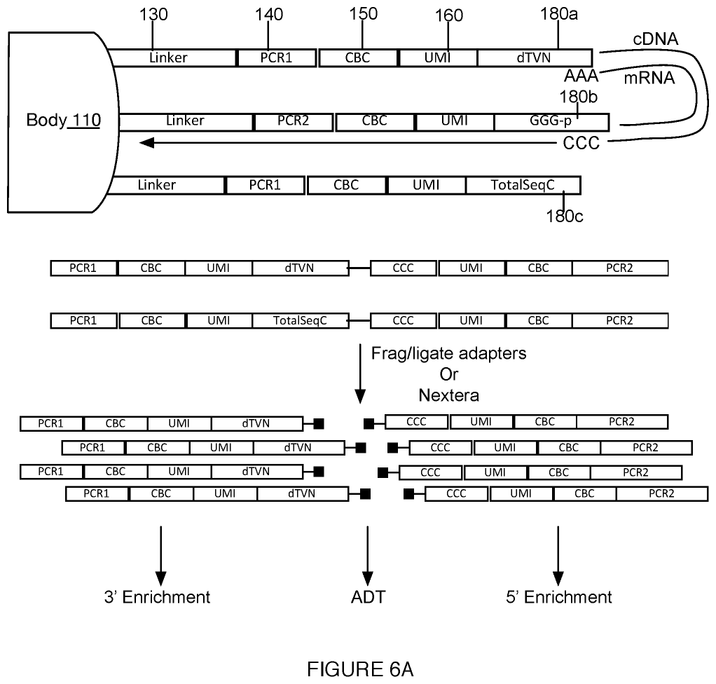

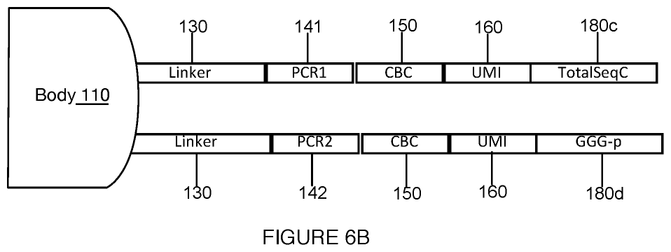

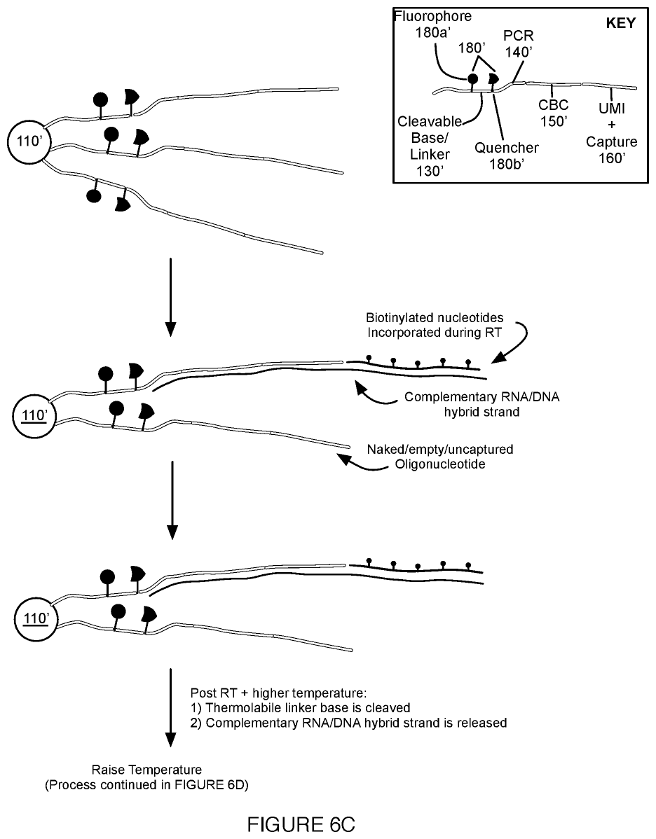

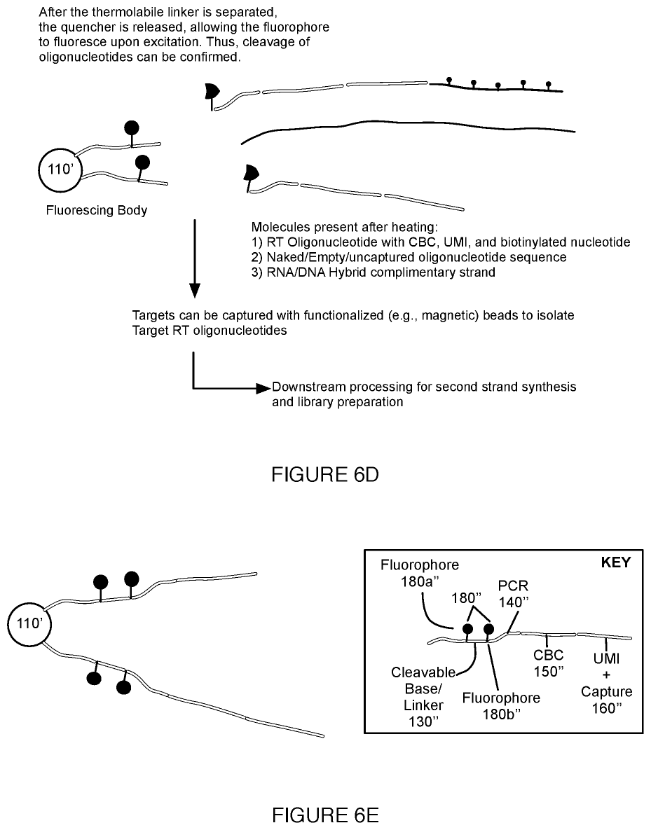

In variations, as shown in FIG. 6A, the active segment 180 of a molecule of the one or more molecules 120 can be adapted for mRNA binding and cDNA synthesis can include one or more of: a first sequence 180a for mRNA binding, such as a PolyT sequence (e.g., a dTVN or TTTTTTTTTTTTTTTTTTTTTTTTTTTTTVN sequence) which enables capture of an mRNA species through PolyA interactions; and a second sequence 180b for interactions with cDNA synthesized from captured mRNA (e.g., a rGrGrG group for interactions with a CCC region added to synthesized cDNA with a reverse transcription enzyme, another group for interactions with another region added to synthesized cDNA with a reverse transcription enzyme, etc.). During operation, an RT enzyme can terminate with addition of a CCC sequence (or other sequence) during cDNA synthesis, then post-denaturation to remove the template mRNA, the cDNA sequence can interact with a GGG containing group (or other complementary group) of the second sequence 180b. where the second sequence is blocked from extension at 3′ end by a phosphate or other suitable blocking group (e.g., C3 spacer, dideoxy nucleotide, etc.,) Specific sequences other than CCC or GGG may be incorporated in the oligonucleotide tags attached to the bead to provide specific molecular interaction functionality and may comprise DNA bases, RNA bases or other groups. As shown in FIG. 6A, the one or more molecules can include a first subset including a first sequence for mRNA binding (e.g., with sequence 180a) and a second subset including a second sequence for cDNA interactions (e.g. with sequence 180b), such that synthesized cDNA product can be captured and purified on-particle at the composition 100 without subsequent purification steps; however, in other variations, the first sequence 180a and the second sequence 180b can alternatively be coupled to different particles. In other variations, the second sequence may not be 3′ blocked and can extend on the cDNA sequence and form a complement to the first strand sequence.

In additional variations as shown in FIG. 6A, the active segment 180 of a molecule of the one or more molecules 120 can be adapted for binding a specific target sequence on an mRNA, DNA, or other nucleic acid target and synthesis can include one or more of: a first sequence 180c for target binding, such as a TotalSeqC capture sequence (e.g., TTTCTTATATGGG), which enables capture of an oligo tag attached to an antibody or such as another target binding oligo (e.g., targeted primer) that targets a specific portion of one or a few mRNA species, a gDNA sequence or other sequence; and a second sequence 180b for interactions with DNA (or cDNA) synthesized from the captured nucleic acid. During operation, an RT enzyme can terminate with addition of a CCC sequence (or other sequence) after templated cDNA synthesis, then post-denaturation to remove the template mRNA, the cDNA sequence can interact with a GGG containing group (or other complementary group) of the second sequence 180b. where the second sequence is blocked from extension at 3′ end by a phosphate or other suitable blocking group (e.g., C3 spacer, dideoxy nucleotide, etc.). Specific sequences other than CCC or GGG may be incorporated in the oligonucleotide tags attached to the bead to provide specific molecular interaction functionality and may be comprised of DNA bases, RNA bases or other groups. As shown in FIG. 6A, the one or more molecules can include a first subset including a first sequence for targeted nucleic acid binding (e.g., with sequence 180c) and a second subset including a second sequence for nucleic acid hybridization (e.g. with sequence 180b), with either a general (e.g., rGrGrG) binding motif or another specific targeted oligo sequence such that the resulting synthesized product can be captured (e.g., between the known sequence elements of 180c and 180b) and purified on-particle at the composition 100 without subsequent purification steps; however, in other variations, the first sequence 180c and the second sequence 180b can alternatively be coupled to different particles. In other variations, the second sequence is not 3′ blocked and can extend directly on the newly synthesized sequence and form a complement to the first strand sequence.

In embodiments, two different oligonucleotide tags present in the same particle as in FIG. 6A can be configured to provide additional advantages. The strand created in FIG. 6A that includes the oligonucleotide plus eDNA, which then continues to be the complement of the second strand ends up capturing the CBC twice, where the second is an inverted complement. In such cases multiple barcode regions originating from the same bead are physically linked provides a means to improve data analysis (i.e., the barcode regions should “match”). However, barcode regions that are non-matching indicates an error (e.g., in vitro recombination if the barcodes are very different, other errors if the differences are only 1-2 bases). As such, this allows one to identify and potentially correct the small errors and thus you have an improved ability to map the cDNA sequence to the correct bead and thus the correct cell. In more detail, such a configuration provides a second point to provide a degree of error correction. Additionally, when the barcodes don't match sufficiently, one can exclude those sequences from analysis (or tentatively assign them to one or the other barcode regions). Another advantage is that one can measure the rate of this type of chimerism in the data and then use those data to correct data that may not be able to measure it directly. For example if one uses beads with only the one barcode region in the same workflow as beads with multiple barcode regions, one could infer a rate of chimerism for the one barcode region scenario from the data generated with the beads conferring two barcode regions per sequence. It is not necessary that both barcodes be identical to match. If the barcodes are constructed to be different but the associations are known the advantage of “matching” is still possible.

In other variations, as shown in FIG. 6B, the active segment 180 of a molecule of the one or more molecules 120 can be adapted for protein tagging and other processes can include one or more of: a third sequence 180c for binding of antibodies (or other protein components) of a target protein, such as an oligonucleotide-antibody binding region (e.g., a TotalSeq™ region) which enables binding of antibodies (e.g., surface antibodies) from a lysed cell; and a fourth sequence 180d for interactions with a generated product derived from captured proteins (e.g., a rGrGrG group for interactions with a CCC region added during synthesis, another group for interactions with another region added during synthesis, etc.). During operation, an RT enzyme can terminate with addition of a CCC sequence (or other sequence) during synthesis, then the synthesized protein product can interact with a GGG group (or other complementary group) of the fourth sequence 180d. As shown in FIG. 6B, the one or more molecules can include a first subset including a first sequence for antibody binding (e.g., with sequence 180c) and a second subset including a second sequence for synthesized product interactions (e.g. with sequence 180d), such that synthesized product can be captured and amplified on-particle at the composition 100 without subsequent purification steps; however, in other variations, the third sequence 180c and the fourth sequence 180d can alternatively be coupled to different particles. Note that some of the purification or enhancement of certain products are enabled by the amplification of certain oligonucleotide sequences over other sequences.

The composition can additionally or alternatively include other active segments in the one or more molecules 120, for performing other processes involving binding/other interactions.

### 2.2.5.1 Cleavable Linkers

For instance, as shown in FIGS. 6C-6E, active segments 180′ can incorporate one or more cleavable fluorophore quencher regions, which can function to enable confirmation of cleavage of oligonucleotides from bodies based upon emitted fluorescent signals. As shown in FIG. 6C (top right), one or more molecules of the composition can include a linker 130′ (as described above) coupling the molecule to the body 110′; an active region 180′ including a cleavable element (e.g., cleavable base or linker) with a fluorophore 180a′ and a quencher 180b′; a PCR handle 140′; a barcode region 150′; and a unique molecule identifier (UMI) 160′ with a capture sequence.

During use, as shown in FIGS. 6C-6D, biotinylated nucleotides can be incorporated during reverse transcription, with generation of a complimentary RNA/DNA hybrid strand on some molecules, and some molecules may not capture any target oligonucleotides. Then, a cleavage signal (e.g., a reaction environment temperature change to 94° C., a reaction environment temperature change to another suitable temperature for a thermolabile linker) produces cleavage of the thermolabile linker of the active region 180′, and release of the complimentary RNA/DNA hybrid strands of the molecules having RNA/DNA hybrid strands. As shown in FIG. 6D, after the thermolabile base/linker is separated, the quencher 180b is released allowing the fluorophore 180a to fluoresce upon excitation. As such, fluorescent signals emitted by the fluorophore 180a can enable confirmation of cleavage of oligonucleotides molecules from the body 110.

In more detail as shown in FIG. 6D, heating results in multiple molecules present in the reaction environment: 1) reverse transcribed oligonucleotides comprising barcode regions 150′ and unique molecule identifiers 160′ with biotinylated nucleotides; 2) naked/empty/uncaptured oligonucleotide sequences; and 3) RNA-DNA hybrid complementary strands. Then, with removal of the liquid phase from the reaction environment, combination of the liquid phase with separation particles (e.g., streptavidin magnetic beads, as described in applications incorporated by reference) followed by separation (e.g., by magnetic force) allows the reverse transcribed oligonucleotides to be isolated for downstream processing and second strand synthesis, with library preparation, as described in U.S. application Ser. No. 16/867,235 filed on 5 May 2020 and U.S. application Ser. No. 16/906,337 filed on 19 Jun. 2020, which are each herein incorporated in its entirety by this reference.

While thermolabile mechanisms are described, the active region 180′ can additionally or alternatively include other cleavable mechanisms whereby products can be detected to confirm cleavage. For instance, the active region 180′ can additionally or alternatively include photocleavable regions, chemically cleavable regions, enzymatically cleavable regions, or regions cleavable by another suitable mechanism.

Furthermore, as described above, the reverse orientation of the fluorophore 180a′ and the quencher 180b′ can be implemented, in order to monitor cleavage and/or capture with emitted fluorescent signals.

In related variations, the active region 180′ can alternatively include a fluorophore, where the fluorophore acts as both a fluorophore and quencher. In particular, when the density of fluorophores on a bead is high enough for self quenching, the removal of some fluorophores from the bead will result in an increase in the total fluorescence even when no specific quencher molecule is included. The cleavage can thus be monitored by an increase in fluorescence (e.g., fluorescence from a bead or fluorescence from a well containing beads and/or released fluorophores in the supernatant) even if the number of beads and number of fluorophores being monitored remains unchanged.

Still alternatively, in another variation of the active region 180′, the quencher 180b may not be a dark quencher but rather another fluorophore (e.g., FRET partner) that affects signals detected from the reaction during operation. For example, the active region 180 can incorporate a first fluorophore (e.g., Fluorescein) on the portion configured to remain on the body 110 post-cleavage, and a second fluorophore (e.g., TAMRA) configured to be released by cleavage, which would result in quenching of the fluorescein signal from the first fluorophore when in close proximity, but an increase in the signal when the oligonucleotide with the second fluorophore is released. Furthermore, the signal from the second fluorophore could be monitored in both cleaved and uncleaved configurations.

In one alternative configuration shown in FIG. 6E, the composition can be configured for direct quantitation of beads (e.g., with both full length and cleaved molecule quantitation). In more detail, one or more molecules of the composition can include a linker 130″ (as described above) coupling the molecule to the body 110″; an active region 180″ including a cleavable element with a first fluorophore 180a″ (e.g., Fluorescein, Cy3 etc.) and a second fluorophore 180b″ (e.g., TAMRA,Cy5, Cy7), and additional elements configured as required for the specific use for example, a PCR handle 140″; a barcode region 150″; and a unique molecule identifier (UMI) 160″ with a capture sequence. Such a configuration can be used for direct quantitation of cleaved portions of the composition post-cleavage, where the composition components can be visualized (e.g., by fluorescent microscopy, by fluorescent reading apparatus) using different wavelength regimes to alternately detect both the uncleaved elements (where FRET partners remain in close proximity) and the cleaved elements (where FRET partners are separated and no longer interact) or preferentially detect only one of the two species. The same or similar composition can be used quantification without visualization (e.g., for shelf-life testing). Furthermore, such a composition can be used with bodies 110 composed of a hydrogel, where the hydrogel material used for the body 110 is translucent and does not autofluoresce.

However, other configurations or combinations of configurations described can be envisioned.

### 2.2.6 Molecule-Molecular Scissors

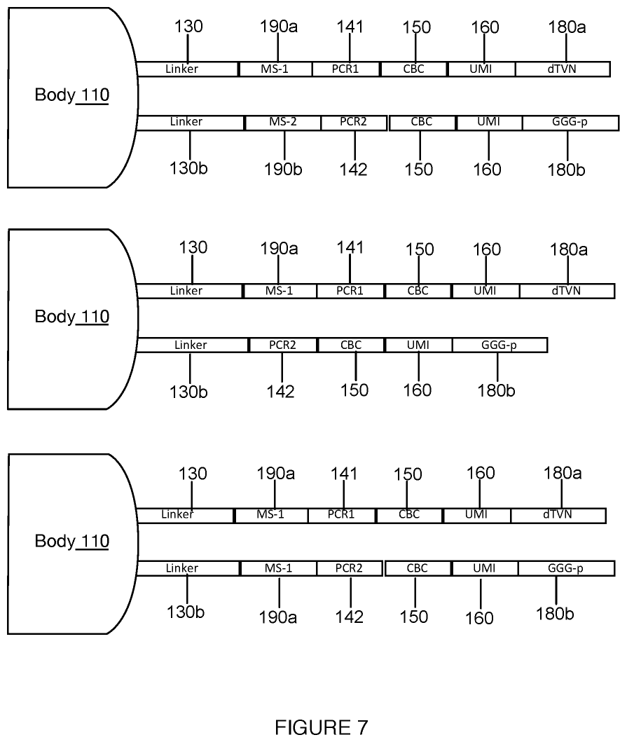

As shown in FIGS. 1 and 7, variations of a molecule of the one or more molecules can additionally or alternatively include one or more optional molecular scissor region(s) 190, which function to enable controlled cleaving of products or other target molecules from the one or more molecules 120 (e.g., post-synthesis, post-reaction, post-generation of product, at a certain point during processing of biological material, etc.). In relation to embodiments, variations, and examples described, molecular scissors broadly include not only the specific USER enzyme blend from NEB, but also restriction enzymes, Zinc finger nucleases, talons, aptamers, transposases, RnaseH, CRISPR enzymes and other molecules that have the ability to recognize specific oligonucleotide (e.g., natural or unnatural) sequences and cut at a specific location of the sequence. In variations, the molecular scissors can be single-stranded or double stranded. In variations, the molecular scissor region 190 is preferably positioned along the oligonucleotide molecule at a region (e.g., immediately distal to the linker) where cleavage will not damage or render unusable desired product. However, the molecular scissor region(s) 190 can alternatively be positioned in another suitable manner. FIG. 7 (top) depicts an example where a unit of the composition includes a first molecular scissor region 190a positioned immediately distal to a first linker 130a along a first oligonucleotide molecule for mRNA capture, and a second molecular scissor region 190b positioned immediately distal to a second linker 130b along a second oligonucleotide molecule for capture of a synthesized cDNA product. This example allows for controlled cleavage of the mRNA capture oligonucleotide separately from the cDNA targeting oligonucleotide. FIG. 7 (middle) depicts an example where a unit of the composition includes a first molecular scissor region 190a positioned immediately distal to a first linker 130a along a first oligonucleotide molecule for mRNA capture, and a second oligonucleotide molecule for capture of a synthesized cDNA product. This example allows for controlled cleavage of the mRNA capture oligonucleotide. FIG. 7 (bottom) depicts an example where a unit of the composition includes a first molecular scissor region 190a positioned immediately distal to a first linker 130a along a first oligonucleotide molecule for mRNA capture, and another instance of the first molecular scissor region 190a positioned immediately distal to a second linker 130b along a second oligonucleotide molecule for capture of a synthesized cDNA product. This example allows for simultaneous cleavage of the mRNA capture oligonucleotide and the cDNA targeting oligonucleotide.

In relation to mRNA binding-cDNA synthesis reactions, the molecular scissor(s) can be configured to be used for cleavage of product pre or post-denaturation to remove mRNA. As such, the molecular scissor region(s) 190 can be used to remove both mRNA-cDNA products, target mRNA, and/or synthesized cDNA products (without mRNA).

In one example embodiment, double stranded specific molecular scissors can be implemented, such that strands are released only after polymerase extension or reverse transcription or similar processes have completed the second strand. In this manner, unreacted products can be washed away, and then completed products can be selectively released and recovered without background contamination from the one or more molecules 120 or other portions of the composition 100. In an alternative variation to the composition shown in FIG. 7, the bottom molecule can be omitted providing functionality described above. In another variation, a molecule having primers as the active portions could provide desired functionality.

Furthermore, in alternative embodiments, the one or more molecules 120 and/or other portions of the composition 100 can include regions designed for controlled cleavage of oligonucleotide sequences and/or other products using other mechanisms (e.g., photocleaving, thermal cleaving, chemical cleavage, etc.).

### 2.2.7 Composition Variation-Functional Molecule Coupled to a Substrate

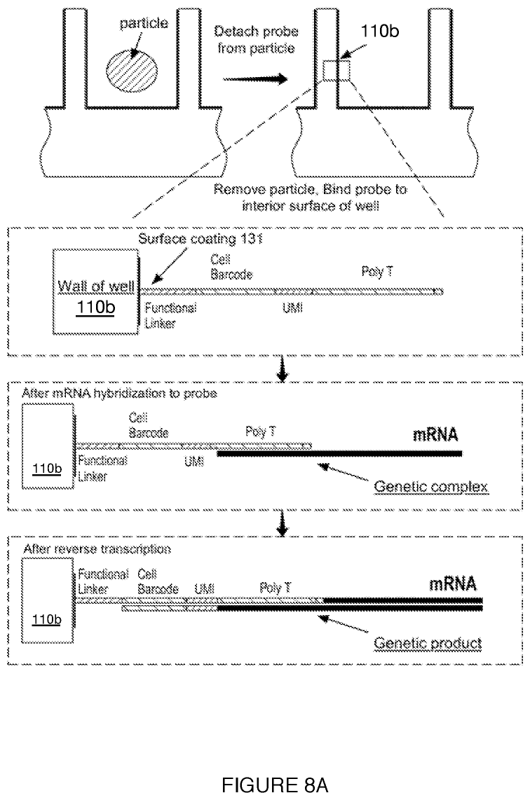

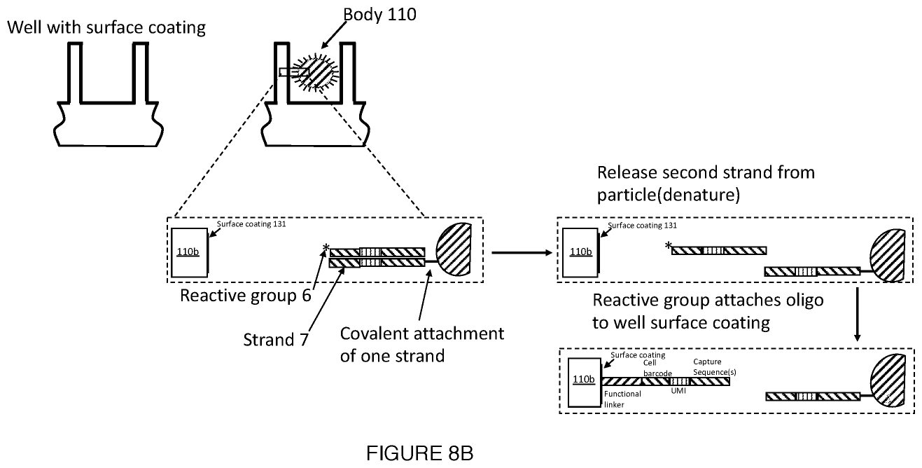

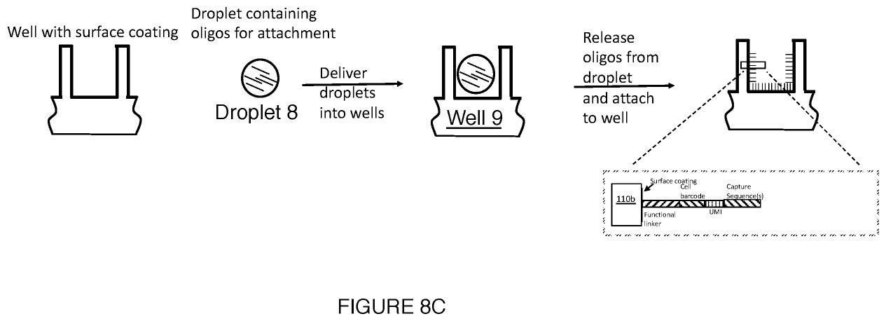

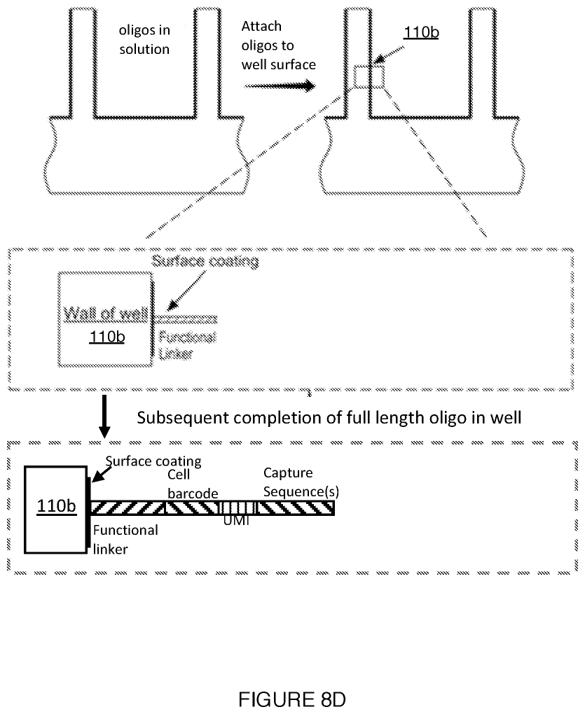

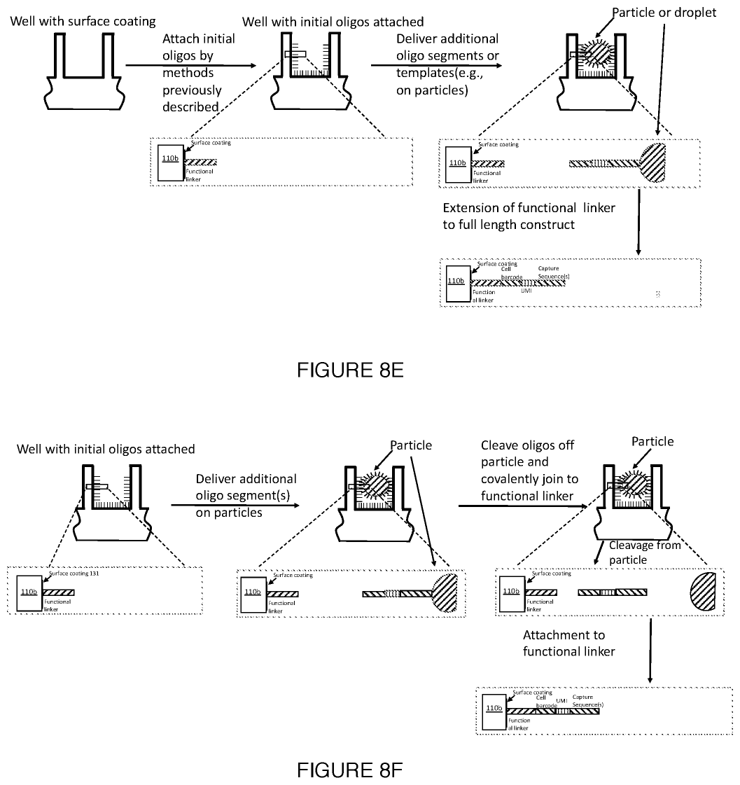

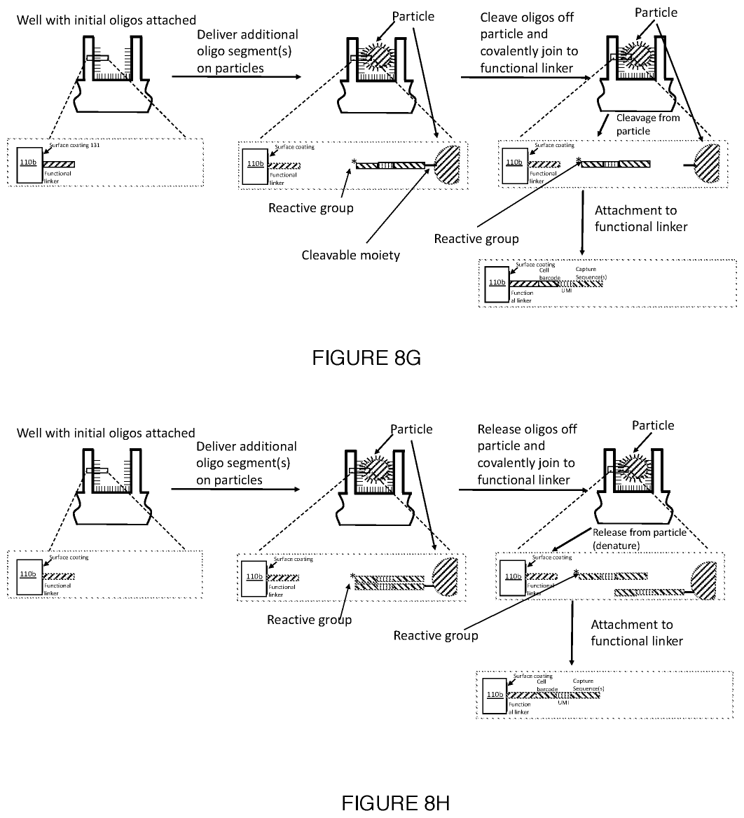

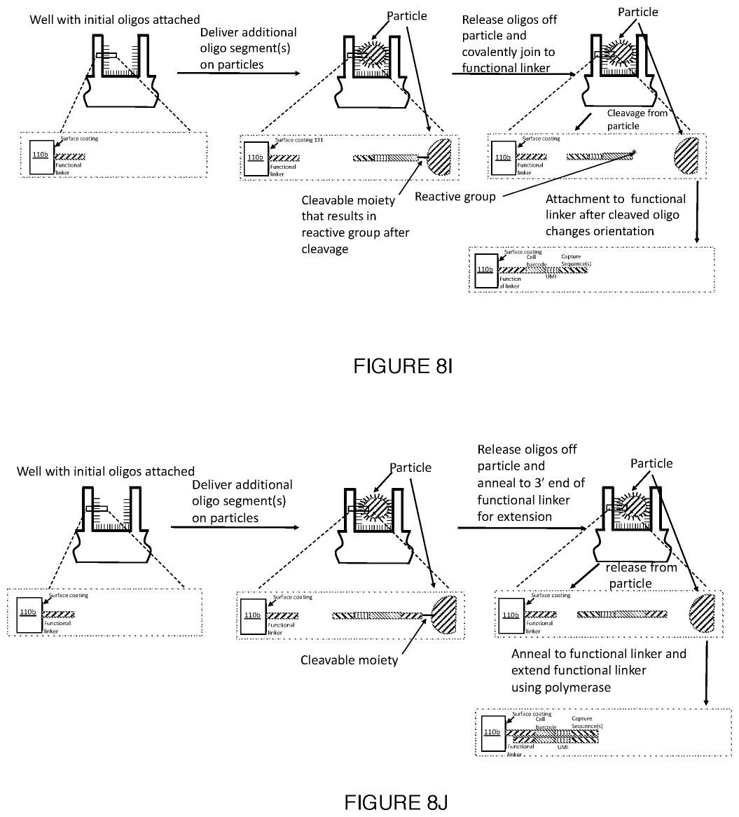

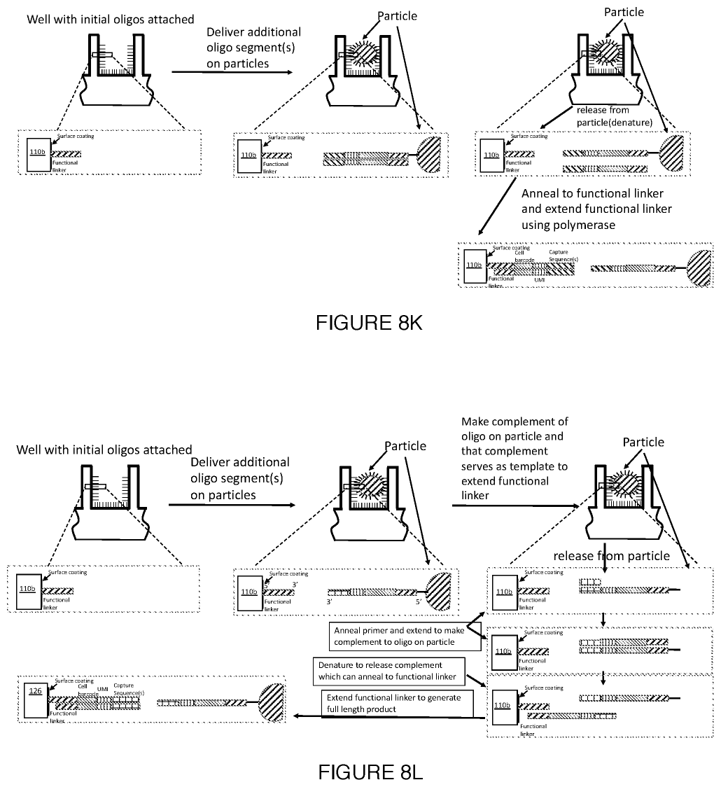

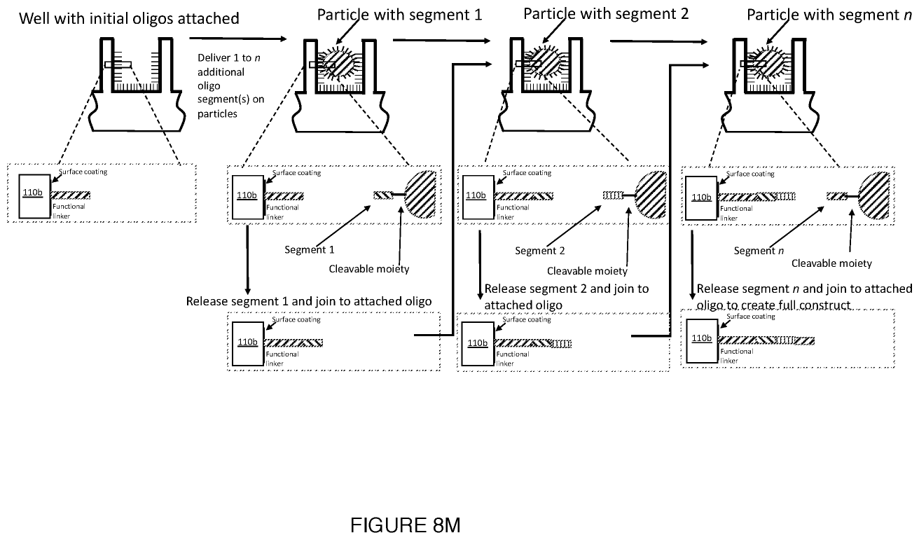

As shown in FIGS. 8A-8C, variations of the composition can be configured for attachment of one of more molecules 120 to a substrate 110b (e.g., as a wall of a chamber, a lid covering a chamber, a protusion that protrudes into a chamber, etc.) used to capture and/or process target materials (e.g., from single cells). Variations of compositions and processes can further be adapted from methods and compositions described in U.S. Pat. No. 10,3891,492, issued 27 Aug. 2019, which is herein incorporated in its entirety by this reference.

In more detail, as shown in FIGS. 8A and 8B, particle-compositions can be configured to deliver functionalized oligonucleotide molecules to the substrate 110b (e.g., wall of a reaction chamber), where the molecules coupled to the particle bodies include reactive groups 6 (e.g., at terminal ends) configured to attach the oligonucleotide to a surface coating 191 of the substrate. In a non-limiting example, the surface coating can include an acrylamide or similar compound(s) and the functional linker attached to the oligonucleotide can include an acrydite modification. As such, attaching the oligonucleotide to the well surface may include polymerizing a plurality of acrylamide and acrydite molecules. In some embodiments, the acrylamide polymer can include crosslinking agents (e.g., Bis-acrylamide), or a reversible crosslinking agent (e.g., [Bis(acryloyl) cystamine], BAC). In some embodiments the polymer matrix can be polymerized in such a way that the oligonucleotide is directly attached to the wall of the well through covalent bonds. In other embodiments the attachment may be indirect. For instance, in one embodiment, the oligonucleotide may be attached by incorporation into a matrix without being directly attached to the wall surface. In this configuration, crosslinks due to the polymerization with BAC are intact and the oligonucleotides remain functionally attached to the wall, but upon reduction of the BAC, the crosslinking is destabilized and a plurality of oligos are then released from the surface into solution. Other example surface coating chemistry and/or functional linker chemistry can be implemented, with respect to the configuration shown in FIG. 8A, and in subsequent configurations shown in FIGS. 8B-8M.

As shown in FIG. 8B, the reactive groups 6 of a strand can be paired with a complementary strand 7 through a hybridized oligonucleotide with covalent attachment to the body 110, and release of the strand with the reactive group 6 from the body 110 can prepare it for attachment to the substrate 110b. With respect to transferring full-length oligonucleotides from a body 110 (e.g., particle 110) shown in FIGS. 8A and 8B to a well surface using the complement of the oligonucleotide attached to the particle, the complement can be constructed (e.g., outside of the well, inside of the well) using a primer with a reactive moiety at the 5′ end (e.g., which can be performed in bulk on multiple bodies/beads), with addition of the beads to wells, followed by denaturing to release complementary oligonucleotides and bind those oligonucleotides to well. Implementation of biotin/streptavidin could provide desired binding results with a number of rounds of denaturing (e.g., from 1 to 5), such that oligonucleotides reannealed in first round can come off in subsequent rounds and bind to available streptavidin at the surface of the substrate 110b.

As shown in FIG. 8C, full-length oligonucleotides for attachment can be delivered in droplets 8 into wells 9, with release of the oligonucleotides from the droplets for attachment to the substrate 110b (i.e., well surface). The droplets can be liquid in air (e.g., delivered by a liquid handling subsystem), or bounded by various materials (e.g., such as in an emulsion, such as an aqueous solution bounded by oil with or without surfactants or other materials, etc.). The droplets may be fully liquid, or can alternatively be composed of a hydrogel. For water in oil droplets, the oligonucleotides could be released by addition of detergents or chemicals that break the emulsion. One non-limiting example is aqueous droplets bound by oil that forms a solid (e.g., wax) structure at lower temperatures, but reverts to fluid at normal biological temperatures.

Alternatively, FIGS. 8D-8M depict variations of attachment processes for coupling and/or building full-length oligonucleotides at the surface(s) of a substrate 110b.

In a first variation, as shown in FIG. 8D, common stub oligonucleotides can be provided in solution and attached to the substrate 110b (e.g., wall surface), and then built out using a suitable process (e.g., using particles, beads, droplets, etc.) from the substrate 110b to generate full-length oligonucleotides.

FIG. 8E depicts one such variation of sequential building from the surface of the substrate 110b, where initial stub oligonucleotides are attached to surfaces within wells as previously described, with delivery of additional oligonucleotide segments or templates (e.g., on particles) within the wells to extend attached oligonucleotides to full-length functional oligonucleotides.

FIG. 8F depicts a mechanism by which attached oligonucleotides can be extended. In more detail, initial stub oligonucleotides attached to surfaces within wells can be extended by delivery of additional oligonucleotide segments on particles, with cleavage (e.g., by chemical means, by thermal means, by photocleaving means, etc.) of such additional oligonucleotide segments from the particles and subsequent joining of the cleaved oligonucleotide segments to functional linkers of the stub oligonucleotides at the well surface.

FIG. 8G depicts a first variation of the mechanism shown in FIG. 8F, where the additional oligonucleotide segments initially coupled to particles include a reactive group configured to attach to a corresponding functional linker upon cleavage from the particle by denaturing. In examples, the reactive group could include a 5′ phosphate for ligation, but can alternatively include an alkyne or azide for click chemistry, or can still alternatively include another reactive group (e.g., carbamate, etc.). According to FIG. 8G, reactive groups/functional linkers can be positioned at 5′ or 3′ orientations depending upon type of reactive group/functional linker chemistry (e.g., 3′ OH configured to react with 5′ phosphate on functional linker). Furthermore, oligonucleotides can be single or double stranded, where an example of a double stranded oligonucleotide with a reactive group is shown in FIG. 8H.

FIG. 8I depicts an alternative variation of the mechanisms shown in FIGS. 8G and 8H, whereby cleavage of a cleavable moiety coupling the oligonucleotide to the particle produces a reactive group that subsequently attaches to the functional linker at the well surface. Again, as shown in FIG. 8I, 5′ and 3′ orientations are not specifically called out. In one non-limiting example, the reactive group could be a 5′ phosphate generated after cleavage, where 5′ phosphate reacts with a functional linker at 3′ end of the oligonucleotide attached to the well surface. In one such example, the on particle oligonucleotide could be constructed with 5′ end attached to the particle and contain a dU residue or abasic site that is cleaved by treatment with uracil DNA glycosylase, followed by a lyase enzyme (e.g., endonuclease III, endonuclease VIII) that cleaves the backbone, resulting in a 5′ phosphate. The cleaved product is then ready to be ligated to an available 3′ OH (e.g., 3′ OH at 3′ end of the oligonucleotide attached to the wall surface). In operation, the attachment to the functional linker can implement a splint to facilitate ligation. In variations, the functional linker can be configured as a partially double stranded construct to act as the splint, the oligonucleotide on the particle could be a double stranded product cut on both strands (e.g., by two dU bases offset) to yield a desired overhang, or an additional oligonucleotide could be added separately to act as a splint.

FIG. 8J depicts an alternative variation of the mechanisms shown in FIGS. 8G-8I, where cleavage of a cleavable moiety coupling the oligonucleotide to the particle releases the oligonucleotide from the particle for annealing to the 3′ end of a functional linker, followed by extension using a polymerase. In variations, shown in FIG. 8J (bottom right), oligonucleotides can remain attached to the particle when well geometry, deformability of particle, or density of functional linkers is such that it is not necessary to release the oligonucleotides from the particle.

FIG. 8K depicts an alternative variation of the mechanisms shown in FIGS. 8G-8J, where single-stranded oligonucleotides are released from the particle with subsequent annealing to the 3′ end of the functional linker at the well surface for extension using a polymerase. In variations, shown in FIG. 8K (bottom right), oligonucleotides can remain attached to the particle when well geometry, deformability of particle, or density of functional linkers is such that it is not necessary to release the oligonucleotides from the particle.

FIG. 8L depicts an alternative variation of the mechanisms shown in FIGS. 8G-8K, where a complement of the oligonucleotide on the particle is constructed and the complement serves as the template to extend the functional linker at the well surface. In more detail, the complement can be constructed by annealing a primer at the particle oligonucleotide with extension to form the complement, following by denaturing to release the complement from the particle. Then, the functional linker can be extended to generate a full length oligonucleotide at the well surface.

FIG. 8M depicts an example mechanism by which additional oligonucleotide segments can be added to a functional linker coupled to the well surface.

In more detail, 1 to n additional segments can be attached to a running build of an oligonucleotide at the well surface, by sequentially cleaving oligonucleotide segments from particles and attaching them to the running build. In relation to the methods of FIG. 8M, any of the preceding methods for attachment can be used serially, alone, or in combination. For instance, each oligonucleotide segment can be ligated on or attached by click chemistry, each could be added by extension after hybridizing a template, or some oligonucleotide segments could be added by extension and others by ligation.

Methods and configurations shown in FIGS. 8A-8M can, however, include other steps or elements, some of which are described in more detail in the following sections.

### 3. Specific Examples of Compositions-ATAC Sequencing, Molecular Scissors, Restriction Sites

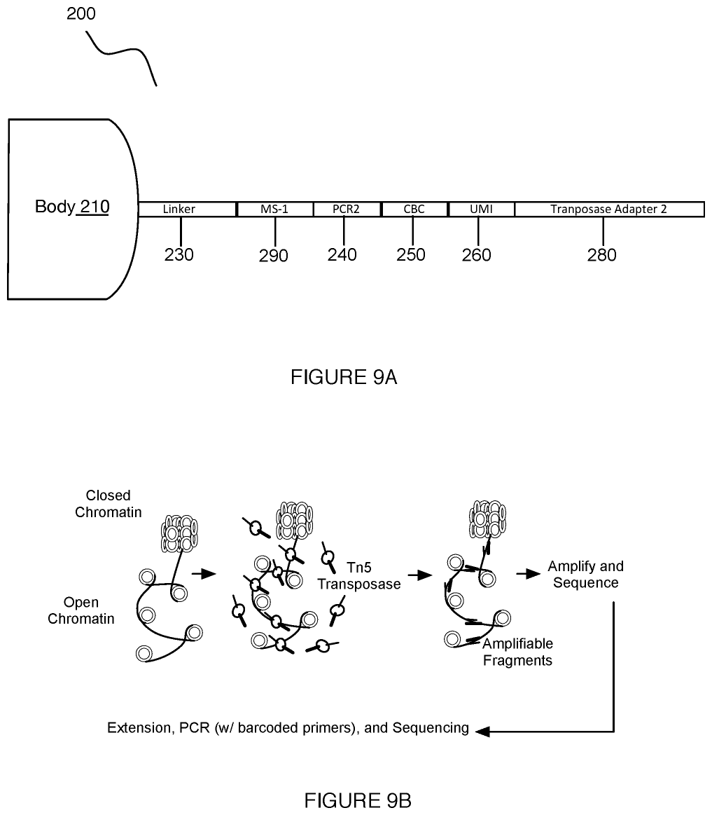

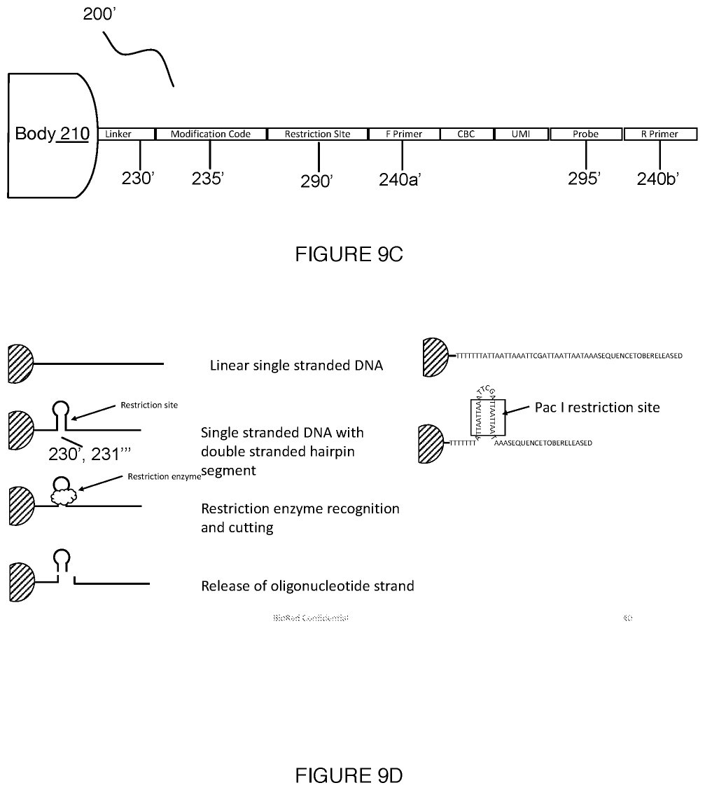

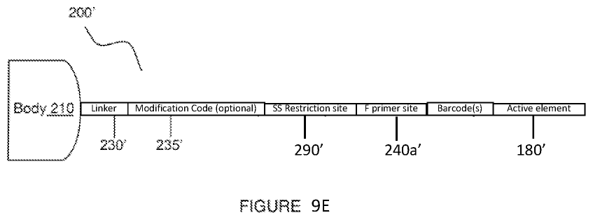

As shown in FIGS. 9A and 9B, variations of a molecule of the one or more molecules 120 can be configured for Assay for Transposase-Accessible Chromatin using sequencing (ATAC-seq), in order to assess chromatin accessibility associated with a genome (e.g., for epigenomic analysis). As shown in FIG. 9A, an example of a composition 200 can include a body 210, a linker 230 coupled to the body, a first molecular scissor region 290 coupled to the linker, a PCR primer 240 coupled to the first molecular scissor region 290, a barcode region 250 coupled to the PCR primer 240, a UMI 260 coupled to the PCR primer 240, and an active segment 280 including a sequence complementary to transposase adaptor (e.g., Tn5 transposase 1, Tn5 transposase 2) for ATAC-seq coupled to the UMI 260. This configuration is configured to accomplish an initial extension reaction, where the other transposase adaptor is used for downstream PCR enrichment of insertion events associated with the first transposase adaptor.

As shown in FIG. 9B, such a composition 200 can be configured for cutting of DNA sequences about chromatin segments with extension by addition of adaptors (and barcodes linked to the transposase adaptor) at each end of each fragment, followed by amplification and sequencing.

Variations of the example shown in FIG. 9A can be configured in another suitable manner. For instance, in a second configuration, the one or more molecules can include a one or more molecules including a linker 230 coupled to the body, a first molecular scissor region 290 coupled to the linker, a PCR primer 240 coupled to the first molecular scissor region 290, a barcode region 250 coupled to the PCR primer 240, a UMI 260 coupled to the PCR primer 240, and an active segment 280 including a first transposase adaptor (e.g., Tn5 transposase-1) coupled to the UMI 260; and a second one or more molecules including a linker 230 coupled to the body, a first molecular scissor region 290 coupled to the linker, a PCR primer 240 coupled to the first molecular scissor region 290, a barcode region 250 coupled to the PCR primer 240, a UMI 260 coupled to the PCR primer 240, and an active segment 280 including a second transposase adaptor (e.g., Tn5 transposase-2) coupled to the UMI 260. This configuration is configured to perform extension and PCR enrichment on the same particle of the composition 200.

In an alternative variation shown in FIG. 9C, the composition 200′ can be configured to include cleavable elements 235′ and 290′which can be used to controllably release oligonucleotides from the body. In more detail, restriction enzymes can be used to specifically cleave DNA, but require a double stranded segment to cut; however, methods described herein often utilize single stranded nucleic acids. As such, to use restriction enzymes, a second nucleic acid often needs to be added to a single-stranded molecule to form a double stranded element for targeted cleavage. This process can produce complications and can result in incomplete cleavage. The composition 200′ can be configured to encode at least one cleavage sites, where the one or more molecules can include a linker 230′ (e.g., long flexible linker, such as a spacer 18 (HEG) sequence providing length and flexibility to bend) coupled to the body, a single stranded sequence encoding a restriction site 290′ (e.g., a type II restriction endonuclease, a type I restriction endonuclease, a type IIG restriction endonuclease, a type IIP restriction endonuclease, a type IIS restriction endonuclease, a type III restriction endonuclease; a type IV restriction endonuclease), and optionally a modification code region 235′ (e.g., an internal deoxyuridine modification code), a forward primer 240a′, a reverse primer binding site 240b′, and an optional fluorescent probe target 295′ (e.g., (FAM)-labeled 5′ nuclease probe, another probe). In the variation shown in FIG. 9C, the oligonucleotide molecules are depicted as linear strands pointing away from the surface of the body, and where the restriction endonuclease requires dsDNA in an antiparallel orientation. Furthermore, the oligonucleotide molecules can take on various confirmations which allow oligonucleotides in proximity to each other (e.g., a first molecule of composition 200′ and a second molecule of composition 200′) to form anti-parallel double stranded constructions, at least transiently in the region of the restriction enzyme recognition sequence, thereby forming complete restrictions sites despite the lack of obvious homology other than the palindromic restriction site sequence. As such, after cleavage using the restriction site 290′ oligonucleotides in solution can be detected, sampled, and quantified using various assays (e.g., by qPCR). In a specific example, the restriction site 290′ includes a BamHI type II restriction endonuclease derived from Bacillus amyloliquefaciens, where the endonuclease has the capacity for recognizing short sequences (e.g., 6 bp) of nucleic acids and cleaving them at a target site. However, other restriction endonucleases can be used as described above.

During experimentation according to an example, untreated beads with ostensibly ss oligonucleotides showed the highest number of molecules released by BamHI cleavage (e.g., approximately twice the number compared to treatment where double stranded products were created by hybridization of a reverse primer and extension by polymerase), and beads with ssDNA and denatured with sodium hydroxide shortly before restriction digestion showed lower cleavage indicating that the cleavage is dependent upon the double stranded state with time requirements for reannealing.

In these variations, the molecule(s) form correct double stranded motifs by transient hybridization between different oligo strands (i.e., they do not form hairpin or other secondary structures within a single strand). Furthermore, no sequences that would complete restriction site are in the rest of a respective molecule strand indicating that intermolecular interactions are required. Furthermore, use of a BamHI restriction site is not only palindromic, but also GC rich to facilitate cleavage; however, other restriction sites can be used although the efficiency of cleavage may vary. In more detail, ssDNA can form loop structures with only a handful of bases, and often can assume a “random coil” configuration, but the linker length and flexibility of the linker region 230′ play a role in getting oligonucleotide pairs to match up to enable targeted cleavage. Furthermore, in these embodiments, it is not required that both strands be attached prior to every cleavage. For instance, Bam HI both strands will be cleaved with the same resulting products due to the manner in which the restriction endonuclease cut, but a missing base will not completely inhibit cleavage; thus, one cleaved oligonucleotide could hybridize with an uncleaved oligonucleotide and induce a second cut (e.g., nick) in the previously uncleaved strand, but without the need for the addition of exogenous complementary strands. As such, density of oligonucleotides coupled to the body plays a role in rate of reactions, but is not strictly required to enable cleavage.

Another specific example of a cleavable linker is shown in FIG. 9D, in which a cleavable linker region 230″ can be used to controllably release oligonucleotides from the body. The composition shown in FIG. 9D builds a sequence feature 231″ into the oligo, where the sequence feature forms a hairpin structure that will, at least transiently, generate a double stranded element (e.g., Pac I restriction site) containing the restriction enzyme recognition/cut site. As such, a temporary double stranded element forms for target cleavage in an intramolecular fashion, thereby enabling release of the corresponding oligonucleotide strand.

The segments of the molecule can, however, additionally or alternatively include other suitable segments as described, and/or be coupled to the body 210 in another suitable manner. As non-limiting examples, the restriction site 290′ of FIGS. 9C and 9E and the cleavable linker element of FIG. 9D can be used to provide controlled cleavage elements for the other compositions described herein (e.g., as the molecular scissors section 190 of composition 100 depicted in FIG. 1, the molecular scissors section 290 of construct 200, the cleavable linker element shown in FIG. 6C, or in other compositions where a cleavable element are indicated or beneficial).

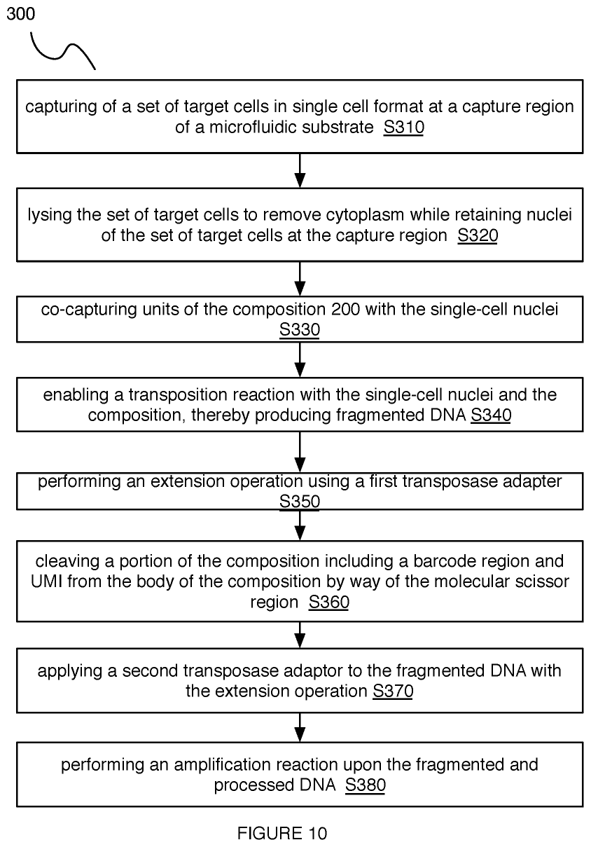

In one example, an embodiment of the composition 200 can be implemented in a method 300 for single-cell ATAC sequencing, where, as shown in FIG. 10, the method 300 includes: capturing of a set of target cells in single cell format at a capture region of a microfluidic substrate S310; lysing the set of target cells to remove cytoplasm while retaining nuclei of the set of target cells at the capture region S320; co-capturing units of the composition 200 with the single-cell nuclei S330; enabling a transposition reaction with the single-cell nuclei and the composition, thereby producing fragmented DNA S340; performing an extension operation using a first transposase adapter S350; cleaving a portion of the composition including a barcode region and UMI from the body of the composition by way of the molecular scissor region S360; applying a second transposase adaptor to the fragmented DNA with the extension operation S370; and performing an amplification reaction upon the fragmented and processed DNA S380.

Variations of the method 300 can further include library cleanup and next generation sequencing loading steps.

Variations of the method 300, can however, be implemented in another suitable manner (e.g., using another capture and processing platform, etc.).

### 4. Manufacturing

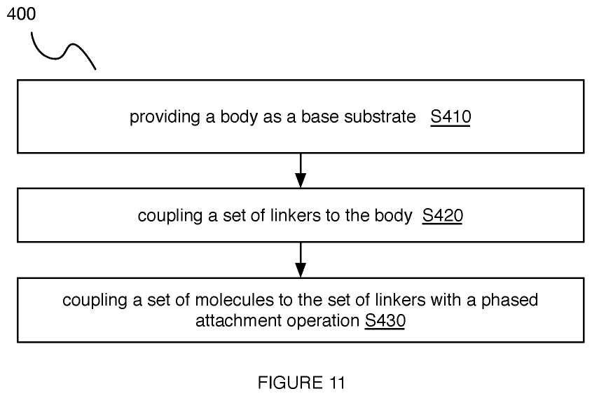

As shown in FIG. 11, a method 400 for generating a composition includes: providing a body as a base substrate S410; coupling a set of linkers to the body S420; and coupling one or more molecules to the set of linkers with a phased/sequential attachment operation S430. In embodiments, a variety of molecular biological reactions (e.g., ligation or polymerase extension) or chemical synthesis methods (e.g., click-chemistries) can be utilized to manufacture long (>50 bp long) oligonucleotide molecules to have very well defined sequences with minimal error rates (e.g., with less than 5% errors, with less than 1% errors, with less than 0.5% errors). In some examples, these can involve templated reactions where the template used to define the sequence is not incorporated directly into the final product. In other examples, the reactions can be untemplated or conducted in a manner such that the template does become incorporated. The oligonucleotides can be built up from component monomer units or by addition of partial or complete sequences. In some examples the units added may be partially or completely single stranded. In other embodiments the units added are partially or completely double stranded. In some embodiments the units added are largely double stranded but only one of the strands becomes covalently linked to the body and/or linker. In some embodiments the template strands and/or the units that are added undergo purification or quality control checks prior to use in the attachment so that the final product has reduced error rates by reducing the errors present in the individual units. In some cases, the method of manufacture of the individual units may inherently assure reduced error rates (e.g., by using short oligonucleotide units). In some embodiments and variations of FIG. 10, the second to last step (e.g., coupling a set of linkers to the body S420 could be optional). For instance, one could potentially have the linker attached to each of the molecules in the set in step S430.

The method 400 functions to efficiently create a composition that allows for processing, separation, and retrieval of target material from a sample, according to one or more benefits described in Section 1 above. The method 400 can produce compositions with complex oligonucleotide structures in a phased-attachment manner, that reduces the compounding error associated with base-by-base oligonucleotide attachment methods (e.g., phosphoramidite based oligonucleotide synthesis). The method 400 can also produce compositions that provide simplification of library preparation processes, by inclusion of molecular adaptors specific to sequencing platforms (e.g., Illumina™ adaptors, etc.). The method 400 can thus be used for manufacturing of functionalized particles in a scalable manner, and in a manner that provides quality control and improvements in the amount of recoverable product.

In embodiments, the method 400 can produce embodiments, variations, and examples of the compositions 100 and 200 described above. However, portions of the method 300 can be adapted to produce other related compositions.

Block S410 recites: providing a body as a base substrate, which functions to provide a base substrate for attachment of functional molecules specific to various processes. As noted above, the base can be provided as a contiguous body or can alternatively be provided as a cluster of smaller bodies. In either continuous or clustered form, Block S410 can include coupling of functional groups (e.g., amines, hydroxyl groups, silanol groups, etc.) to the body in order to facilitate subsequent attachment of linker molecules to surfaces of the body.

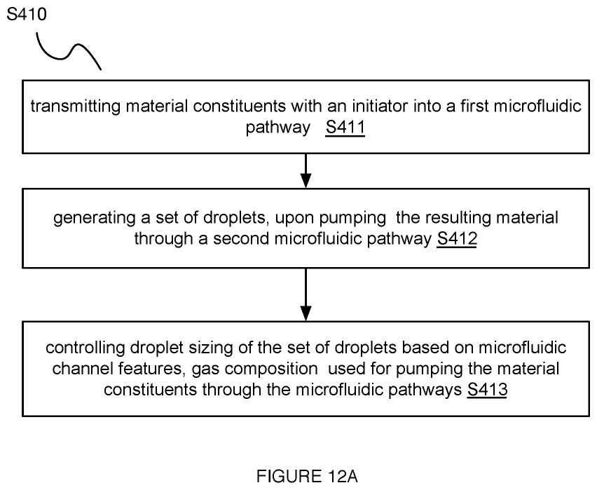

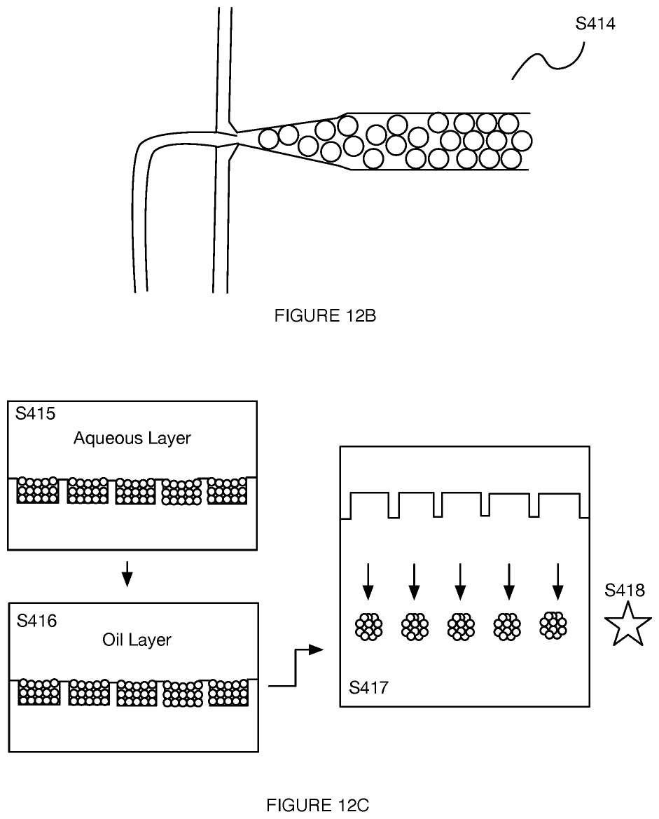

In an alternative variation, as noted above, Block S410 can include aggregating a set of smaller bodies to form the body. In a first variation, as shown in FIG. 12B, Block S410 can include creating droplets of unpolymerized and/or uncross-linked material S414 using a microfluidic channel, whereby the material undergoes polymerization and/or crosslinking in droplet state to form a set of smaller bodies. According to Block S414, the material can be flowed through a microfluidic channel at a desired rate and through an opening having desired morphology, into a medium (e.g., oil, etc.) in order to produce droplets of a desired size. Polymerization can then be achieved through chemical or other means. Similarly, crosslinking can be achieved using one or more of: a photoactivated method, a chemical method, a heat-induced method, and/or any other suitable method.

In another alternative variation, as shown in FIG. 12C, Block S410 can include distributing a set of smaller bodies across a set of wells of a substrate in pre-polymerized aqueous solution S415, with an aqueous layer of fluid over the set of wells. Then, Block S410 can include replacing the aqueous layer of fluid with a separation layer (e.g., a layer of low density oil, such as silicone oil) S416, to separate clusters of smaller bodies within the set of wells. Then, Block S410 can include inverting the substrate S417 or otherwise displacing the clusters of smaller bodies from the set of wells (e.g., with centrifugal force, with other applied force), where surface tension within the separation layer of fluid promotes spherical morphology of each of the set of clusters within the separation fluid. Variations of Block S310 can further include polymerization and/or crosslinking of the clusters of smaller bodies S318 (e.g., at another region within the separation layer of fluid, outside of the separation layer of fluid). In variations, Block S416 can include photopolymerization (e.g., with UV light, with light of another wavelength, etc.) or chemical polymerization of each of the set of clusters of smaller bodies. Block S316 can additionally or alternatively include crosslinking (e.g., crosslinking by irradiation, chemical crosslinking, heat-based crosslinking, oxidative crosslinking, etc.).

Other variations of Block S410 can, however, involve additional or alternative steps for formation of a set of clustered smaller bodies having suitable surface chemistry (and/or core material features, such as magnetism), in order to provide a substrate for functionalization with oligonucleotides.

In a first variation, Block S410 can include generating base substrates in the form of beads, where the beads are composed of a polymer that dissolves in controlled environments. In a specific example, the beads can be composed of a polyacrylamide material processed from an acrylamide solution (e.g., 40% v/v acrylamide, another percentage of acrylamide), Bis(acryloyl) cystamine (e.g., 0.8% w/v BAC, another percentage of BAC, deionized water, and a buffer (e.g., a buffer composed of Tris-HCL, NaCl, KCl, EDTA, Triton X-100, and water, another suitable buffer, etc.), where the polyacrylamide beads are configured to polymerize with Ammonium persulfate (e.g., 10% APS, another percentage of APS) and Tetramethylethylenediamine (TEMED) under low oxygen conditions (e.g., under Argon gas) and later to dissolve in the presence of a reducing agent such as dithiothreitol (DTT).

In this variation, as shown in FIG. 12A, producing beads according to Block S410 includes: transmitting material constituents with an initiator into a first microfluidic pathway S411; generating a set of droplets with resulting material of S411, upon pumping (e.g., with a pressurized gas pump) the resulting material through a second microfluidic pathway (e.g., a 14 μm focusing channel terminating at a 500 μm collection volume) with TEMED provided to the oil phase during collection S412; and controlling droplet sizing of the set of droplets based on microfluidic channel features, gas composition (e.g., argon, other gas) used for pumping the material constituents through the microfluidic pathways S413. In a specific example of S410-S413, a pressurized pump (e.g., with pressured argon to pressurize and remove air from the pump chamber in order for hydrogels to polymerize), with control of pressure and flow rate was coupled to a first microfluidic chip including the first fluidic pathway and a second microfluidic chip including the second fluidic pathway, where quality and size of the droplets formed was monitored using an X-Y stage and high-speed camera mounted to a microscope controlled with a flow control center. In the example, formed polyacrylamide droplets were washed with a buffer composed of Tris-HCL, NaCl, KCl, EDTA, Triton X-100, and water, and placed in a storage solution of Tris Tween-20, where the formed droplets had a mean diameter of 22.75 μm (e.g., in aqueous solution with swelling), with a standard deviation of 1.62 μm. In the example, the droplets were dissolvable in 0.1M DTT at a 1:1 volume ratio, within 30 seconds.

In a variation of the example associated with FIG. 12A, the formulation of the polyacrylamide beads was adjusted by reducing the amount of acrylamide and adding acrylamide-tagged (e.g., acrydite modified) oligonucleotides, to provide approximately 109 oligonucleotides per bead. In some variations the oligos were further modified (e.g., with a fluorophore or other modification) for fluorescent tagging and detection applications. In more detail, the beads can be composed of a polyacrylamide material processed from an acrylamide solution (e.g., 40% v/v acrylamide, another percentage of acrylamide), Bis(acryloyl) cystamine (e.g., 0.8% w/v BAC, another percentage of BAC, deionized water, acrydited oligonucleotides (e.g., 250 UM acrydited fluorescein amidite (FAM) oligos having an acrydited site proximal to a first end and a FAM site proximal a second end), ammonium persulfate solution (e.g., 10% w/v APS, another percentage of APS), and a buffer (e.g., a buffer composed of Tris-HCL, NaCl, KCl, EDTA, Triton X-100, and water, another suitable buffer, etc.), where the FAM-tagged polyacrylamide beads are configured to polymerize with Tetramethylethylenediamine (TEMED) and dissolve in a solution of dithiothreitol (DTT). In the example of fluorescent-tagged beads, formed droplets had a mean diameter of 20.39 μm (e.g., in aqueous solution with swelling), with a standard deviation of 1.25 μm. In the example, the droplets were dissolvable in 0.1M DTT at a 1:1 volume ratio (e.g., with imaging at 0 seconds, 30 seconds, 90 seconds, and 5 minutes), where fluorescent signals were indicative of the dissolving process. In this non-limiting example, the DTT breaks the disulfide crosslinks present due to the BAC elements thereby releasing the smaller bodies (e.g., polyacrylamide linked oligos) from the spherical beads. The smaller bodies are of a size that can readily diffuse through the solution. However, variations of this non-limiting example can also be implemented.

Block S420 recites: coupling a set of linkers to the body, which functions to control spacing and density of a set of oligonucleotide molecules coupled to the body to produce functionalization of the composition. In embodiments, the linker can be an embodiment, variation, or example of the linker 130 described above; however, the linker can be another suitable linker.

In variations involving asymmetric linkers (e.g., linkers having branches of different lengths or linkers of similar length but with different functional or protecting groups), Block S420 can include building a first oligonucleotide segment off of a first branch of the asymmetric linker while protecting a second branch with a second protecting group, and separately building a second oligonucleotide segment off of the second branch of the asymmetric linker while protecting the first branch with a first protecting group (and deprotecting the second branch) S425. Variations of Block S425 can, however, be configured to operate without using a linker, or by coupling an oligonucleotide that has already been synthesized, to an attachment site of the composition.

Block S430 recites: coupling one or more molecules to the set of linkers with a phased/sequential attachment operation, which functions to reduce compounding error and lot-to-lot variability associated with typical chemical synthesis of oligonucleotide chains. In more detail, Block S430 functions to provide a method that involves fewer addition events to produce lower compounding error, in order to create higher accuracy oligonucleotide molecules, more control over design of the molecules, and higher efficiency of synthesis, in relation to the amount of usable full-length product (e.g., over 97% usable product). In some embodiments it further serves to confine the incomplete products to discrete units that are larger than a single base which provides advantages that may keep the partial products from participating in downstream workflows, and facilitates data analysis that can distinguish manufacturing errors from artefacts of downstream processes which can improve subsequent data analysis.

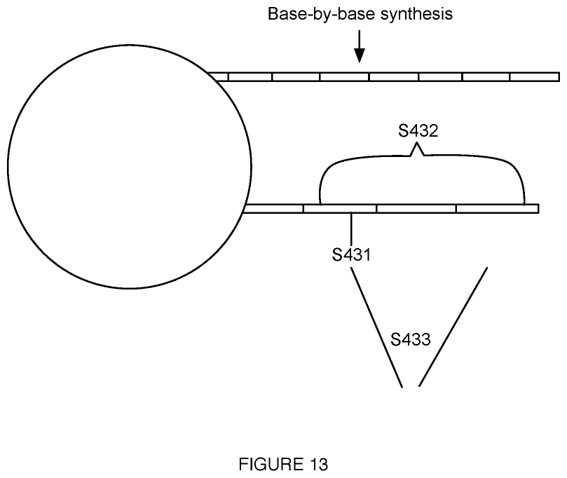

As shown in FIG. 13, In variations, Block S430 can include generating a set of sub-segments (e.g., in parallel, in series) of a desired oligonucleotide molecule S431 configured for reactions described above. Then, Block S430 can include assembling the set of sub-segments into the desired oligonucleotide molecule S432 as a full-length product with reduced error. In some variations, Block S430 can include purifying units of the set of sub-segments S433 in order to further reduce error in assembly, where purification can include full purification processes and/or desalting steps. Additionally or alternatively, some variations can include purification-associated steps after assembly of the desired oligonucleotide molecule; however, some variations of the method S430 can omit purification steps associated with Block S433. In variations, the phased attachment method of Block S430 involves generation of sub-segments that are from 5-30 bases in length, which are then assembled; however, in alternative variations, the phased attachment method of Block S430 can involve generation of sub-segments of other suitable lengths.

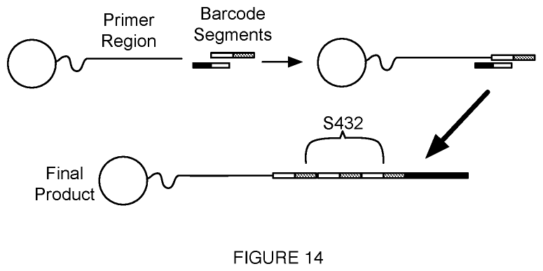

In relation to barcode segments or other segments described above, as shown in FIG. 14, a specific example of Block S430 can include generation of barcode segments (e.g., segments approximately 20 bases long), where, as shown in FIG. 14, the barcode segments are selected from a group of barcode sequences with 96-384 versions. However, another suitable number of barcode sequence versions can be generated with non-naturally occurring sequences of suitable length.

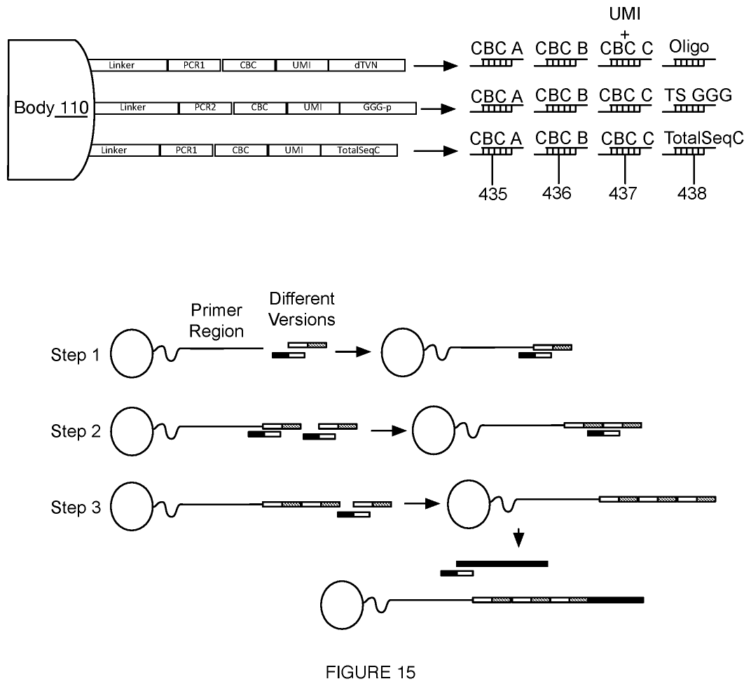

In the specific example, 3 segments of barcode sequences can be generated with unique overhangs (e.g., having associated identifiers), where the overhangs can be used to facilitate correct assembly of the oligonucleotide molecule in a desired order. For instance, as shown in FIG. 15, a first barcode sequence 435 can include an overhang for coupling with a second barcode sequence 436 having an overhang for coupling with a third barcode sequence and unique molecule identifier 437 with an overhang for coupling to an active group 438 (e.g., Oligonucleotide TVN, TS GGG, TotalSeq C, etc.). The assembled barcode segments can be coupled to a precursor molecule (e.g., linker coupled to primer) coupled to the body provided in Block S410 or coupled to a precursor molecule in another manner.

In still more detail regarding the specific example, a precursor of the composition can be constructed with a body (e.g., bead) coupled to a linker (e.g., C18 linker) coupled to an oligonucleodie comprising a primer binding site (e.g., TSO primer) followed by a set of bases (e.g., 8 thymine bases). Then, a first barcode segment with overhangs on each side of the first barcode segment can be pre-hybridized and then coupled to the precursor of the composition with an appropriate ligase enzyme. Subsequent barcode segments with overhangs can then be coupled to the running build of the barcode region, until a desired barcode region length is achieved. For each step of assembly of barcode segments, complementary segments comprising a detection portion (e.g., fluorophore segment) can be tagged onto the current segment being added, where detection of the detection portion (e.g., by an optical detection process) can be used for quality control at each step of phased attachment. However, quality control at each phase of the phased attachment method can be performed in another suitable manner, or omitted.

Still alternative variations of Block S430 can include performing a synthesis operation configured for single-base addition of nucleotides to form an oligonucleotide product. In a specific example of the alternative variation, chemical synthesis involves addition of nucleotide bases, base-by-base, to a linker (e.g., C18 linker) to produce a full length product. Furthermore, variations of the method 400 can include a hybrid approach, whereby a portion of an oligonucleotide molecule (e.g., linker and primer segments) are formed by base-by-base synthesis, and remaining portions of the oligonucleotide molecule are formed by a phased attachment approach involving assembly of shorter sub-segments of oligonucleotides.

The method 400 can additionally or alternatively include other suitable steps. For instance, variations of the method 400 can include steps associated with manufacturing, scale-up, and quality control in order to improve efficiency of generating usable product, including one or more of: performing a reaction with a ligase (e.g., NEB-M0202M) in a controlled environment (e.g., with a desired concentration per number of particles being generated) in order to couple generated oligonucleotide segments; providing a desired concentration of oligonucleotide material per number of particles being generated; providing a desired reaction volume (e.g., within a container that allows sufficient headroom for wash steps); providing a stabilization reagent (e.g., polyethylene glycol) during manufacturing in order to improve reaction efficiency; implementing a shaking procedure (or other procedure to thoroughly disperse or create uniform product with desired reaction conditions); implementing an incubation procedure (e.g., 16±5° C. or 16±1° C.) during manufacturing of the composition; and performing a suitable number of wash steps. Additionally, variations of the method 400 can exclude certain elements from the manufacturing process such as manufacturing with DTT free ligase and removing DTT from the other reagents in the process, or excluding other potential release agents for the smaller bodies (temperature, chemical, etc.) from the manufacturing process. However, the method 400 can additionally or alternatively include other suitable steps of processes for mass production of units of the composition 100, 200.

### 4.1.1 First Manufacturing Example—Next Generation Barcoded Beads

In one example, the method 400′ can be adapted to create multiple barcode sets on each body, in a manner where a single bead has different combinations of barcode sequences, using a limited (e.g., a few) sets of barcodes combined in known and unique combinations. All of the combinations of barcode sequences on a single bead can be unique to the bead, or can be otherwise configured. As such, the method 400′ can implement a limited set of barcodes combined together in known combinations so that a single manufacturing build results in multiple barcodes (CBC's) per bead in a controlled and predictable manner, such that all the different barcodes can map back to the same bead.

In more detail, each barcode unit can include a barcode unit subsequence having a set of bases (e.g., less than 10 bases, more than 10 bases) and a handle or handles (e.g., one of a set of different ligation handles or one of a set of ligation handles on either end or other handle(s) such as polymerase extension handle(s)), where the barcode unit subsequences can be configured as sets defined primarily by the handle. In variations, the handles can each have between 3 and 15 bases, or another suitable number of bases. Each of the barcode unit subsequences in an assembled set is thus configured with the same handle(s) (e.g., one of a set of different ligation handles), with different sets having other handles of the set of different handles. The number of ligation handles can thus be determined based upon the number of barcode sequences desired per bead and total barcode diversity desired.

In examples, the method 400′ can implement a number of barcode unit subsequences (e.g., 96 barcode units, 384 barcode units, another number of barcode units), along with a set of ligation handles (e.g., 4 ligation handles, less than 4 ligation handles, more than four ligation handles) to achieve a desired level of diversity for the sample(s) being processed and desired number of different barcodes per bead. Each of the sets could have unique barcodes, but alternatively, the same set (e.g., of 96 barcode subsequences, of 384 barcode subsequences, etc.) could be used for all the sets. In one example, 96 barcode unit subsequences with a 7-mer barcode can be implemented with a 4 base ligation handle, where the barcode unit subsequences are selected from four different sets of 96 barcode unit subsequences; however, other numbers of sets of barcode unit subsequences could be used including a single set differentiated in context only by the handle sequences.

Expanding the example, to provide four uniquely different barcode sequences on one bead, the method 400′ can implement a first set having barcode subsequences of XXXXXXX with the ligation handle ATCG, where XXXXXXX is a 7-mer barcode sequence (e.g., one of a set of 96 barcode sequences, one of 384 barcode sequences, one of another number of barcode sequences); a second set having barcode subsequences of XXXXXXX with the ligation handle TCGA, whereX is the 7-mer barcode sequence; a third set having barcode subsequences of XXXXXXX with the ligation handle CGAT, where XXXXXX is a 7-mer barcode sequence; and a fourth set 404′ having barcode subsequences of X with the ligation handle GATC, where is a 7-mer barcode sequence. As such, the ligation handles ATCG, TCGA, CGAT, and GATC are specific to the set, but the subsequences XXXXXX may not be specific to the set. In this example, the specific 4 base ligation handles are different for the first (e.g., ATCG, TCGA, CGAT, and GATC), second (e.g., TCAG, AATC, ATTA, TCCT), third, and 4th ligation reactions associated with an individual bead and are also be different for each set of barcode unit subsequences. As such, this configuration provides 16 different handles across four sets of barcode unit subsequences with 4 ligation events (e.g., the number of handles is a product of the number of sets of barcode unit subsequences and the number of desired ligation events).

During implementation of the method 400′, all of a first set of barcode versions can be provided in a first well, all of a second set of barcode versions can be provided in a second well, and so on, in order to generate uniquely barcoded beads with different barcodes coupled to each bead (e.g., well 1 contains barcode 1 ATCG, barcode 1 TCGA. Barcode 1 CGAT, and barcode 1 GATC; well 2 has barcode 2 ATCG, barcode 2 TCGA. Barcode 2 CGAT, and barcode 2 GATC, etc.). In alternative variations, different barcode versions from each set can be provided in each well as long as each well has one uniquely identifiable barcode from each barcode set (e.g., well 1 has barcode 1 ATCG, barcode 25 TCGA. Barcode 49 CGAT, and barcode 76 GATC; well 2 has barcode 2 ATCG, barcode 33 TCGA. Barcode 82 CGAT, and barcode 25 GATC, or alternatively if each barcode set originates from a different set of 96, for example, well 1 has barcode 1 ATCG, Barcode 97 TCGA, barcode 193 CGAT, and barcode 290 GATC, etc.)

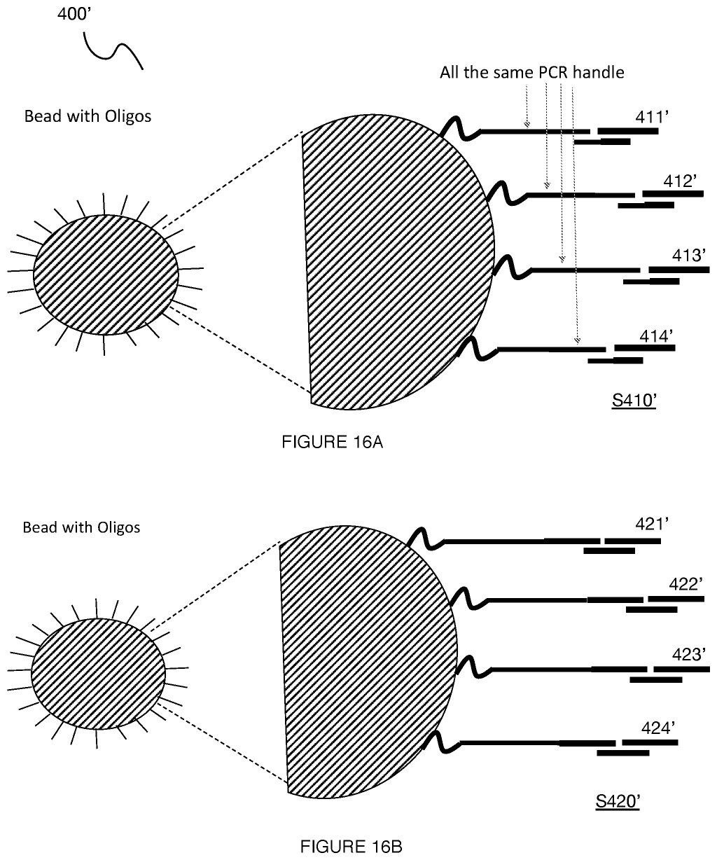

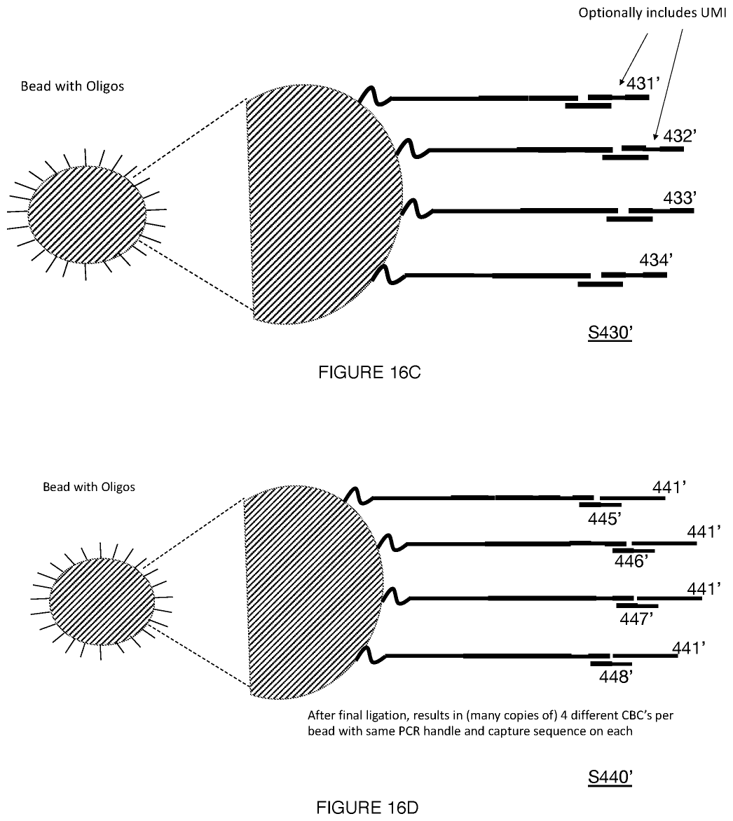

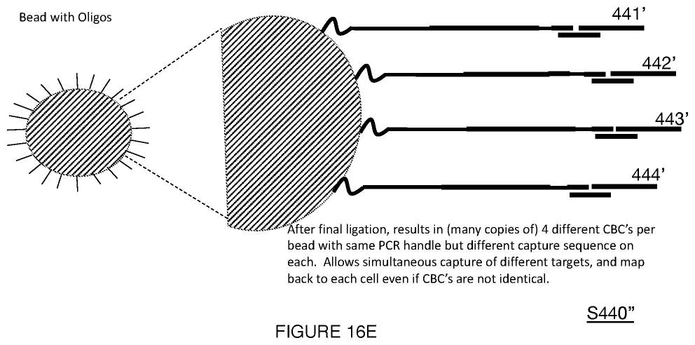

FIGS. 16A-16D depict a sequence of creation of a bead (i.e., body 110′) with four different barcodes, where each individual bead ends up with a set of four uniquely identifiable barcodes (CBCs) after a set of ligation events. As shown in FIG. 16A, the example method 400′ can include: adding a first set of barcode unit subsequences with different ligation handles at a 3′ end S410′, where different barcode unit subsequences 411′, 412′, 413′, 414′ of the first set of barcode unit subsequences are hybridized with splint oligonucleotides 415′ having the same overlap sequence. In relation to step S410′, each of the first set of barcode unit subsequences can be added together to achieve a desired ratio between different units (e.g., 1:1:1:1, non-1:1:1:1 ratio, etc.). The resulting product after the first ligation round would be 4 different oligonucleotide strands (or another suitable number in other variations) on each bead each of which has a different ligation handle. In one variation, the barcode unit subsequences may be identical within a well, with different ligation handles used to distinguish the sets. In another variation, barcode unit subsequences may be different, but known association due to being from same well.

As shown in FIG. 16B, the example method 400′ can include: adding a second set of barcode unit subsequences to corresponding ends of the first set of barcode unit subsequences S420′, where second barcode unit subsequences are shown as 421′, 422′, 423′, 424′ in FIG. 16B. In relation to step S420′, each of the second set of barcode unit subsequences can be added together to achieve a desired ratio between different units (e.g., 1:1:1:1, non-1:1:1:1 ratio, etc.).

As shown in FIG. 16C, the example method 400′ can include: adding a third set of barcode unit subsequences to corresponding ends of the second set of barcode unit subsequences S430′, where third barcode unit subsequences are shown as 431′, 432′, 433′, 434′ in FIG. 16C. In relation to step S430′, each of the second set of barcode unit subsequences can be added together to achieve a desired ratio between different units (e.g., 1:1:1:1, non-1:1:1:1 ratio, etc.). Furthermore, as shown in FIG. 15C, the third set of barcode unit subsequences can optionally include a unique molecular identifier sequence, as described above.

As shown in FIG. 16D, the example method 400′ can include: adding a set of capture oligonucleotides to corresponding ends of the third set of barcode unit subsequences S440′, where the similar capture oligonucleotides are shown as 441′ in FIG. 16D, and different splint oligonucleotides (i.e., 445′, 446′, 447′, 448′) are implemented. While three sets of barcode unit subsequences are described, the method 400′ can include addition of any other suitable number of barcode unit subsequences in order to achieve desired diversity. In relation to the example method 400, the result after 3 (or however many) rounds of ligation with pooling and splitting between rounds is beads with the same barcode diversity we would have with single barcode sequences, but 4 different barcode sequences on each bead. It would be possible to put 4 different capture sequences on these beads using the different ends, and because the barcode unit subsequence associations are known, any barcode sets should match not only at a single barcode position, but across the set of 3 barcode unit subsequences making up an aggregate barcode sequence.

In more detail, if the same capture sequence is applied to all oligonucleotide strands of a particular bead, even as they comprise different composite barcodes, the pool of sequences from any cell will all map to one of a limited whitelist set of barcode subsequences associated with that particular bead allowing better identification of sequencing errors or chimeric sequences. The ligation handles used further correspond to a particular set for all positions of an aggregate barcode sequence aggregated from individual barcode unit subsequences. As such, any crossing of sets could be detected and those sequences flagged. In clinical applications, the ability to have even relatively rare (e.g., greater than 1) captured sequences (e.g., transcripts) confirmed to originate from the same cell because there are multiple different barcodes that ALL map to the same bead (and thus same cell) would greatly improve the certainty of any calls associated with the barcodes, and thus any potential diagnoses. A particular transcript or a set of transcripts associated with aggregate barcode sequence but different UMI's is probable to be different transcripts from a single target cell, but could result from chimeric sequences. As such, mapping to 4 different aggregate barcodes, all of which are associated with a single bead, provides much greater confidence that they originated from a single cell.

An additional benefit of using individual sets of barcode unit subsequences according to the example method 400′ is that the “invariant” ligation handles will now, collectively in association with each single bead, have diversity and thus avoid sequencing flags allowing more cost effective use of the downstream processes.

While three sets of barcode unit subsequences are described, the method 400′ can include addition of any other suitable number of barcode unit subsequences. In relation to the example method 400, the result after 3 (or however many) rounds of ligation with pooling and splitting between rounds is beads with the same barcode diversity we would have with single barcode sequences, but 4 different barcode sequences on each bead. It would be possible to put 4 different capture sequences on these beads using the different ends, and because the barcode unit subsequence associations are known, any barcode sets should match not only at a single barcode position, but across the set of 3 barcode unit subsequences making up an aggregate barcode sequence.

In a variation of the method 400′, as shown in FIG. 16E, the method can include: adding a set of capture oligonucleotides to corresponding ends of the third set of barcode unit subsequences S440″, where capture oligonucleotides correspond to those shown as 441′, 442′, 443′, 444′ in FIG. 16E Step S440″ varies from Step S440′ described above in that after a final ligation step, the resulting composition includes multiple different aggregate barcode sequences (CBCs) per bead with the same PCR handle, but with different capture sequences on each aggregate barcode sequence. As such, this configuration allows simultaneous capture of different targets, with the ability to map back to each cell reliably even if the aggregate barcode sequences are not identical.

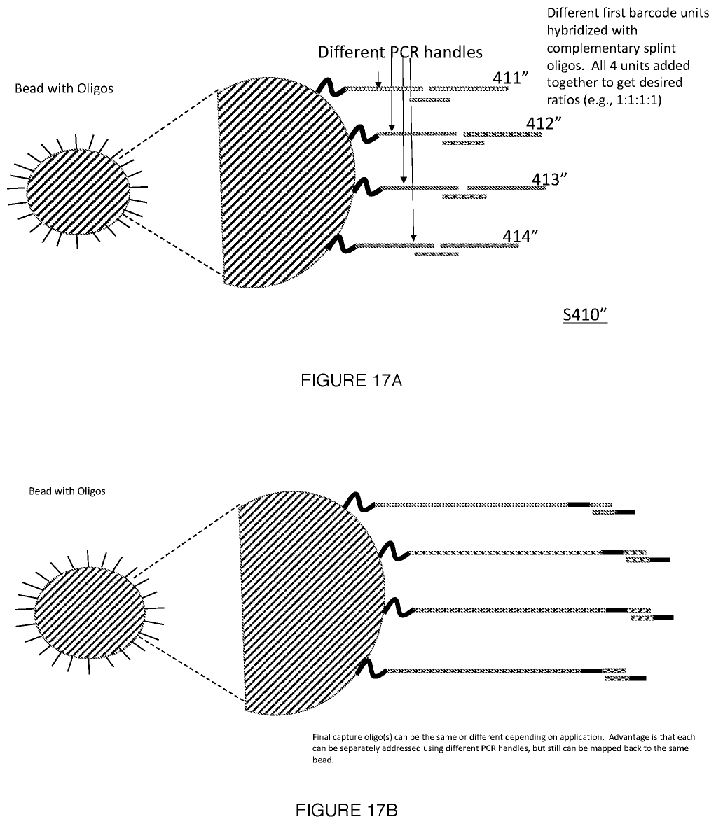

In still another variation of the method 400′, As shown in FIG. 17A, the method can include: adding a first set of barcode unit subsequences with different ligation handles at a 3′ end, with addition to different PCR handles S410″, where different barcode unit subsequences 411″, 412″, 413″, 414″ of the first set of barcode unit subsequences are hybridized with splint oligonucleotides 415″ having complementary overlap sequence. In relation to step S410″, each of the first set of barcode unit subsequences can be added together to achieve a desired ratio between different units (e.g., 1:1:1:1, non-1:1:1:1 ratio, etc.). Then, in a manner similar to that described in relation to Steps S420′ through S440′ described above and shown in FIG. 17B, the method can generate bead compositions where each bead has a different barcode sequences that can be addressed independently due to the different PCR handles applied in Step S410″. In particular, the final capture oligonucleotide(s) can be the same or different depending on application. Furthermore, each can be separately addressed using different PCR handles, but still can be mapped back to the same bead. As such, there can be linkage and association with a particular cell/bead even if samples are processed using different downstream workflows (e.g., after initial capture and extension by reverse transcription or polymerase extension).

As described above, methods 400′ and 400″ are shown to append oligonucleotide sequences to a bead; however, the methods 400′ and 400″ can additionally or alternatively be adapted to incorporate cleavage sites (e.g., molecular scissors, restriction sites, etc.) as described in various variations above. Furthermore, in some applications, the oligonucleotides may be attached to bead by 5′ end and have free 3′—OH group. In other applications, the oligonucleotides may be attached to the bead by 3′ end. In other applications, different barcode sets could include oligonucleotides assembled to potentially have identical sequences after ligation, but are configured in a manner where one barcode set is added by extending the oligonucleotide along a 5′ to 3′ direction, and the other oligonucleotide is extended along a 3′ to 5′ direction.

With respect to steps of the methods 400′ and 400″ described above, keeping the beads in suspension during the ligation is beneficial to the overall ligation and likely to the uniformity of the ligation among beads. The precise speed will vary with the size and shape of the container and the number of beads in the reaction. Associated mixtures were shaken at 1500 RPM in a shaking apparatus in an example; however, other shaking parameters can be implemented. With respect to timing of each ligation step., ligation times of less than 1 hour may reduce the overall ligation efficiency, or require additional enzyme to achieve the same efficiency. In examples, ligation time periods of between 4 hours and 24 hours per ligation were implemented, with incubation at 16° C.; however other ligation times and incubation temperatures can be implemented.

Inherent in the split and pool synthesis approach for bead manufacturing is that beads with incomplete oligonucleotides will be combined together. As such, there is the potential for un-ligated barcodes from one well to become ligated onto oligos on beads that were originally in different wells. This is particularly true when the number of “stubs” (i.e., incomplete oligos attached to bead) is not completely saturated with barcodes. The result would be beads with more than one barcode on the same bead, and this would result in incorrect assignment of sequence data during analysis. This type of contamination would be very undesirable. If the beads (and ligation reaction components) from multiple wells are collected into larger tubes, collection of the beads, followed by pelleting to retain the beads and remove supernatant, followed by washing of the beads, significantly reduces cross-contamination to mitigate the above described effects (e.g., if performed rapidly). Alternatively, for the automation system or when any beads are left in mixed solution at intermediate states, ligation should be inhibitied (e.g, with a stop solution, with heat killing of enzymes, with dephosphorylating the barcode oligonucleotides, with adding blocking oligos, with depleting the ATP from the ligation solution, in another suitable manner). An example stop solution can include EDTA combined with approximately 2× the molar equivalents of Mg++present.

The ideal number of oligonucleotides per bead further vary based on bead composition and final application of use. For instance, improved performance and reduced cost can be achieved for ligations with sub-maximal amounts of barcode oligonucleotides. An example process implemented 850 nanomoles of partially double stranded oligonucleotides in a ligation reaction with approximately 3.5 million beads, or about 0.25 picomols per bead. By reducing the amount of partially double stranded oligonucleotides to 172 nanomoles per 3.5 million beads, or about 50 femtomoles per bead, the cost of manufacture was significantly reduced with improved performance. This example achieved more optimal distribution of oligonucleotides around each bead, resulting in less steric hindrance as adjacent oligonucleotides where steric hindrance would be an issue were ligated at lower rates resulting in a more distributed set of full length oligonucleotides. The amount of ligase also scales with number of beads and with the number of ligation events per bead. In the example, 33,333 cohesive end units per 3.5 million beads were implemented, or about 0.0095 cohesive end units per bead.

Other ligation reaction components that can improve ligation include PEG 6000 to a final concentration of 10% w/v, Mg++ to a concentration of 10 mM (or by replacing up to ˜ 50% of the Magnesium with another divalent cation or with a much larger amount of monovalent cations where monovalent=120*square root of [divalent]). Other ligation reaction components can additionally or alternatively be implemented to produce suitable reaction environments.

Furthermore, while ligation is described in the example methods 400′, 400″, other methods of assembly or extension could be implemented (e.g., templated polymerase extension or chemical attachment, such as click attachment, etc.).

In relation to barcode unit subsequence lists described in relation to the methods above, various example lists can include between 96 (or less) and 932 (or more) barcode unit subsequences. In particular, sets can be configured for greater Hamming distance, Levenshtein distance, or other distance, in order to provide characteristics for easy correctability by post-sequencing analyses. Sets can additionally or alternatively be configured for producing beads with lower total barcode diversity.

However, other suitable configurations and/or numbers of barcode units per list can be implemented.

### 4.1.2 Second Manufacturing Example

In examples, methods for manufacturing may start with multiple wells (e.g., 96 wells), each well containing over 1 million microspheres and one unique oligonucleotide segment attached (e.g., by ligation) to each bead under optimal conditions of time, temperature and shaking and compositions (e.g., enzyme concentration, oligonucleotide concentration, reaction enhancers, molecules to provide crowding). After ligation of the unique oligonucleotide tag to all the particles present in each tube (e.g., 96 tubes), the beads could be washed such that no carryover of products happen after washing when all the beads from 96 tubes would be pooled together (e.g., 1 million beads per tube×96 tubes=96 million beads pooled).

After washing an additional time, the beads are re-distributed into 96 different tubes containing a unique barcoded oligonucleotide segment and then additional reagents added (e.g., ligase, ATP, PEG, reaction enhancers) to continue the second phase of attachment. This process of barcode segment reaction, washing, pooling, redistributing is continued until all the different oligonucleotide segments area added to complete the entire process. The liquid handling process for split-pooling-washing and reaction of beads may be automated in 96 well plates or may be automated in other plate sizes such as 384 well plates or 1536 well plates. The dispensing of reagents in each well may be done by a liquid pipettor or may be done by other methods such as ink-jet-type nozzles, or acoustically ejected from an inverted well plate. The pooling of beads can be done by a pipettor or done by using a specially designed received lid plate that can be placed on the 96 well plate and then the plate-lid assembly inverted and shaken to collect all the beads in the receiver lid plate. Liquid handling operations are designed such that contamination of steps during the entire operations are minimized to prevent any errors to propagate through the entire process. This invention described herein will allow the workflow for manufacturing these barcoded beads to be significantly streamlined. The total number of beads that can be manufactured can be as low as 10 million to as high a 10 billion, with a bead diversity of more than 100,000 (or 1 million or >10 million) different unique combinations.

In some embodiments, the unique oligonucleotides present in each well may include different size fragments in different wells. For clarity, a specific example might be that 32 of the wells might each contain of a partially double stranded construct including of 6 bases providing overlap with the previous segment to facilitate ligation, 7 unique bases that define a barcode segment, and 4 bases to provide overlap with the following segment. An additional 32 wells contain of a partially double stranded construct including the same 6 bases providing overlap with the previous segment, 8 unique bases that define a barcode segment, and 4 bases to provide overlap with the following segment. A third set of 32 wells each contain a partially double stranded construct including the same 6 bases providing overlap with the previous segment, 9 unique bases that define a barcode segment, and 4 bases to provide overlap with the following segment. When used in the manufacturing method described above this would result in full length oligonucleotides that differ in length due to the inclusion of the different length fragments. When sequences are subsequently generated that read through the barcode regions, the barcodes manufactured in this way would have multiple distinct benefits for the sequence generation and analysis that are not present with a typical manufacturing process. In particular, when a plurality of sequences are generated from a plurality of beads, those generated by the above process can have the beneficial attribute that the overlap sections for some or all of the sequences should be identical. They can thus serve as alignment markers and provide other benefits to the analysis such as identifying chimeric molecules, sequencing or manufacturing errors, and other benefits.

The sequencers typically used for these analyses will produce errors and terminate the run, thus failing to collect the desired experimental data, if too large a portion of the sequences all contain the same base at a particular position. As such, inclusion of identical sequences, such as the identical overlap regions described, can be problematic when all of the sequences are the same length. By varying the length of the barcode units preceding the constant regions in the manner described herein, the resulting sequences become offset. While the overlap region or regions may be fundamentally invariant across the plurality of sequences, they are effectively out of phase such that the benefits of identical or near identical markers can be achieved without causing errors in the sequencing process itself. This can be implemented in the described manufacturing process with different numbers of wells or tubes and different configurations of sequence length variation that those used here for illustration as long as they are suitable to provide the dual benefit of working with the constraints of the sequencing instrumentation limitations and providing improved analysis post sequencing.

### 5. Conclusion

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

1-33. (canceled)

34. A composition for single-cell analysis comprising: a dissolvable body comprising a crosslinked polymer; and a plurality of oligonucleotide molecules coupled to the body, each oligonucleotide molecule comprising a barcode region.

35. The composition of claim 34, wherein the crosslinked polymer comprises a reversible crosslinking agent that dissolves in the presence of a reducing agent.

36. The composition of claim 35, wherein the crosslinks comprise disulfide bonds.

37. The composition of claim 36, wherein the reducing agent breaks the disulfide bonds to dissolve the body.

38. The composition of claim 37, wherein the reducing agent is dithiothreitol.

39. The composition of claim 34, wherein the body is formed from smaller bodies held together by the polymer, and wherein dissolution of the polymer releases the smaller bodies to increase accessible surface area.

40. The composition of claim 34, wherein the oligonucleotide molecules are single-stranded and each contains a palindromic sequence, wherein pairs of the oligonucleotide molecules hybridize at their palindromic sequences to form double-stranded restriction sites.

41. The composition of claim 34, further comprising second oligonucleotide molecules coupled to the body, each comprising a second barcode sequence different from the barcode region, wherein the different barcode sequences map to the same cell.

42. The composition of claim 41, further comprising third and fourth oligonucleotide molecules with third and fourth barcode sequences respectively, all mapping to the same cell.

43. The composition of claim 34, wherein the body dissolves within 30 seconds to 5 minutes.

44. A method for processing biological samples comprising: providing dissolvable beads coupled to oligonucleotide molecules having barcode regions, wherein the dissolvable beads comprise a crosslinked polymer; co-capturing the dissolvable beads with cells containing target molecules; binding the target molecules from the cells to the oligonucleotide molecules; and dissolving the beads.

45. The method of claim 44, wherein dissolving the beads comprises adding a reducing agent that breaks crosslinking in the polymer.

46. The method of claim 45, wherein the crosslinked polymer comprises disulfide bonds.

47. The method of claim 46, wherein the reducing agent is dithiothreitol.

48. The method of claim 44, wherein co-capturing comprises encapsulating individual beads with individual cells in droplets.

49. The method of claim 44, wherein the target molecules are mRNA molecules, and the method further comprises reverse transcribing the mRNA molecules using the oligonucleotide molecules as primers to form barcoded cDNA molecules.

50. The method of claim 49, wherein dissolving the beads releases the barcoded cDNA molecules.

51. The method of claim 44, wherein co-capturing comprises capturing individual beads with individual cells in wells of a substrate.

52. The method of claim 44, wherein dissolving occurs within 30 seconds to 5 minutes.

53. The method of claim 44, further comprising monitoring dissolution using fluorescent signals.
